(19) United States
(12) Patent Application Publication
(10) Pub. No.: US 2009/0179551 A1
(43) Pub. Date: Jul. 16, 2009

(54) Organic Electroluminescent Compounds and Display Device Using the Same

(76) Inventors: Hyuck-Joo Kwon, Seoul (KR); Young-Jun Cho, Seoul (KR); Seung-Soo Yoon, Seoul (KR); Bong-Ok Kim, Seoul (KR); Sung-Min Kim, Seoul (KR)

Correspondence Address: CLARK & BRODY, 1090 VERMONT AVENUE, NW, SUITE 250, WASHINGTON, DC, 20005, US

(21) Appl. No.: 12/085,312

(22) PCT Filed: Nov. 22, 2006
(86) PCT No.: PCT/KR2006/004922

§ 371 Date: May 21, 2008

(30) Foreign Application Priority Data
Nov. 22, 2005 (KR) 1020050112046
Nov. 21, 2006 (KR) 1020060115344

(51) Int. Cl. H01J 1/63; C07C 211/00

(52) U.S. Cl. 313/504; 564/378; 564/380; 564/338

(57) ABSTRACT

The present invention relates to novel organic electroluminescent compounds having an indenofluorene or a difluorene skeletal, and a display employing the same as an electroluminescent material. The organic electroluminescent compounds according to the invention exhibit good electroluminescent efficiency and excellent life property, so that an OLED device having very good operative lifetime can be advantageously prepared.

### FIELD OF THE INVENTION

The present invention relates to novel organic electroluminescent compounds represented by Chemical Formula 1 and display devices employing the same as an electroluminescent material.

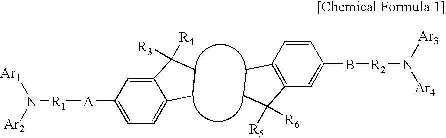

### BACKGROUND OF THE RELATED ART

The most important issue in developing an organic electroluminescent device having high efficiency and long life is development of electroluminescent material of high performance. In view of current development of electroluminescent material, it is the fact that red or blue electroluminescent materials show even lower electroluminescent property as compared to green electroluminescent materials. In order to realize a full color display, three electroluminescent materials (red, green and blue) are employed, and the material having the lowest property among the three materials eventually determines the performance of the entire panel. Thus, development of blue or red electroluminescent material having high efficient and long life is an important matter to enhance the properties of an entire organic electroluminescent device.

As a blue electroluminescent material, diphenylanthracene, tetraphenylbutadiene, distyrylbenzene derivatives and the like have been developed, but the compounds have been known to have low stability of thin film so that they tend to be readily crystallized. Diphenyldistyryl type blue electroluminescent materials having improved stability of thin film where the phenyl group of side chain inhibits crystallization have been developed by Idemitsu [H. Takailin, H. Higashi, C. Hosokawa, EP 388,768 (1990)]. Distyrylanthracene derivatives having improved stability of thin film due to electron withdrawers and electron donors have been developed by Kyushu University [Pro. SPIE, 1910, 180 (1993)].

In addition, DPVBi of Chemical Formula A from Idemitsu Kosan, arylethylene derivatives such as DPVDPAN of Chemical Formula B, dinaphthylanthracene of Chemical Formula C from Kodak Company and tetra(t-butyl)perylene system of Chemical Formula D, as disclosed in EP 1063869 A1 (Idemitsu Kosan Company Limited), Korean Patent Laid-Open 2000-0048006 (Eastman Kodak Company, USA) and Japanese Patent Laid-Open 1996-333569, have been widely used as blue electroluminescent materials.

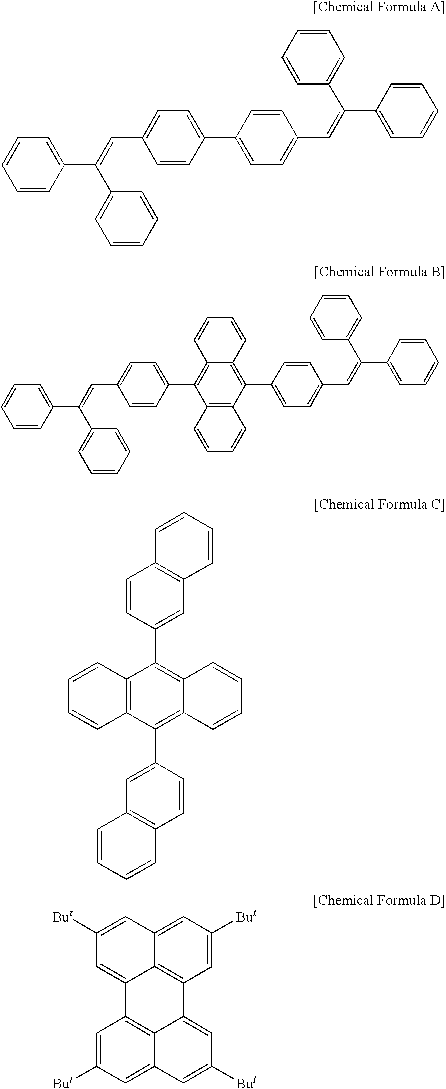

Since DPVBi of Chemical Formula A involves problem of thermal stability having low glass transition temperature of 100° C. or less, DPVDPAN of Chemical Formula B where anthracene is incorporated inside the biphenyl of said DPVBi has improved thermal stability by raising the glass transition temperature to 105° C. The color purity and electroluminescent efficiency as a blue electroluminescent material, however, were not in a quite satisfactory level.

In the meanwhile, blue electroluminescence, when the electroluminescent wavelength is shifted from the present state to a longer wavelength, becomes advantageous in terms of electroluminescent efficiency, but it does not fulfill pure blue color so that it involves difficulties to be applied to a full-colored organic electroluminescent display requiring an electroluminescent material of pure blue color.

Thus, development of novel blue electroluminescent material is an urgent subject in order to develop a blue electroluminescent device or a full-colored electroluminescent device, because conventional blue electroluminescent compounds have lower electroluminescent efficiency than that of other colors.

### DISCLOSURE

### Technical Problem

The present invention noticeably improves the properties of a host which serves as a solvent or an energy carrier in electroluminescent materials as compared to the conventional materials. The object of the invention is to provide organic electroluminescent compounds having excellent electroluminescent efficiency and very good operative life. Another object of the invention is to provide an organic electroluminescent device containing the novel organic electroluminescent compound.

### Technical Solution

The present invention relates to a novel organic electroluminescent compound represented by Chemical Formula 1:

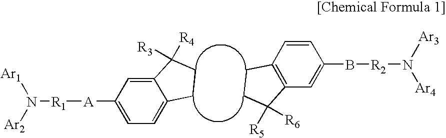

wherein,

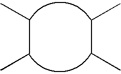

represents

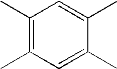

or

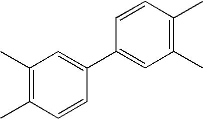

A and B independently represent a chemical bond,

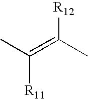

or,

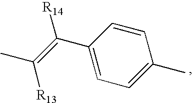

R₁ and R₂ independently represent an aromatic ring or a fused multi-cyclic aromatic ring having two or more aromatic rings,

R₃ through R₆ independently represent a linear or branched C₁-C₂₀ alkyl group with or without halogen substituent(s), a C₅-C₈ cycloalkyl group, or an aromatic group with or without halogen substituent(s),

R₁₁ through R₁₄ independently represent a hydrogen, a C₁-C₈ alkyl group, a C₅-C₈ cycloalkyl group or an aromatic group with or without halogen substituent(s),

Ar₁ through Ar₄ independently represent an aromatic ring or a fused multi-cyclic aromatic ring having two or more aromatic rings: and a display device employing the same as an electroluminescent material.

In the Chemcal Formulas of the present invention, it is referred to as “chemical bond” when there is no element in A or B but R₁ or R₂ is simply connected.

The organic electroluminescent compounds according to the present invention are advantageous in that they can be easily prepared in a high yield with high electroluminescent properties.

The compounds of Chemical Formula 1 according to the present invention include the compounds represented by Chemical Formulas 2 to 6:

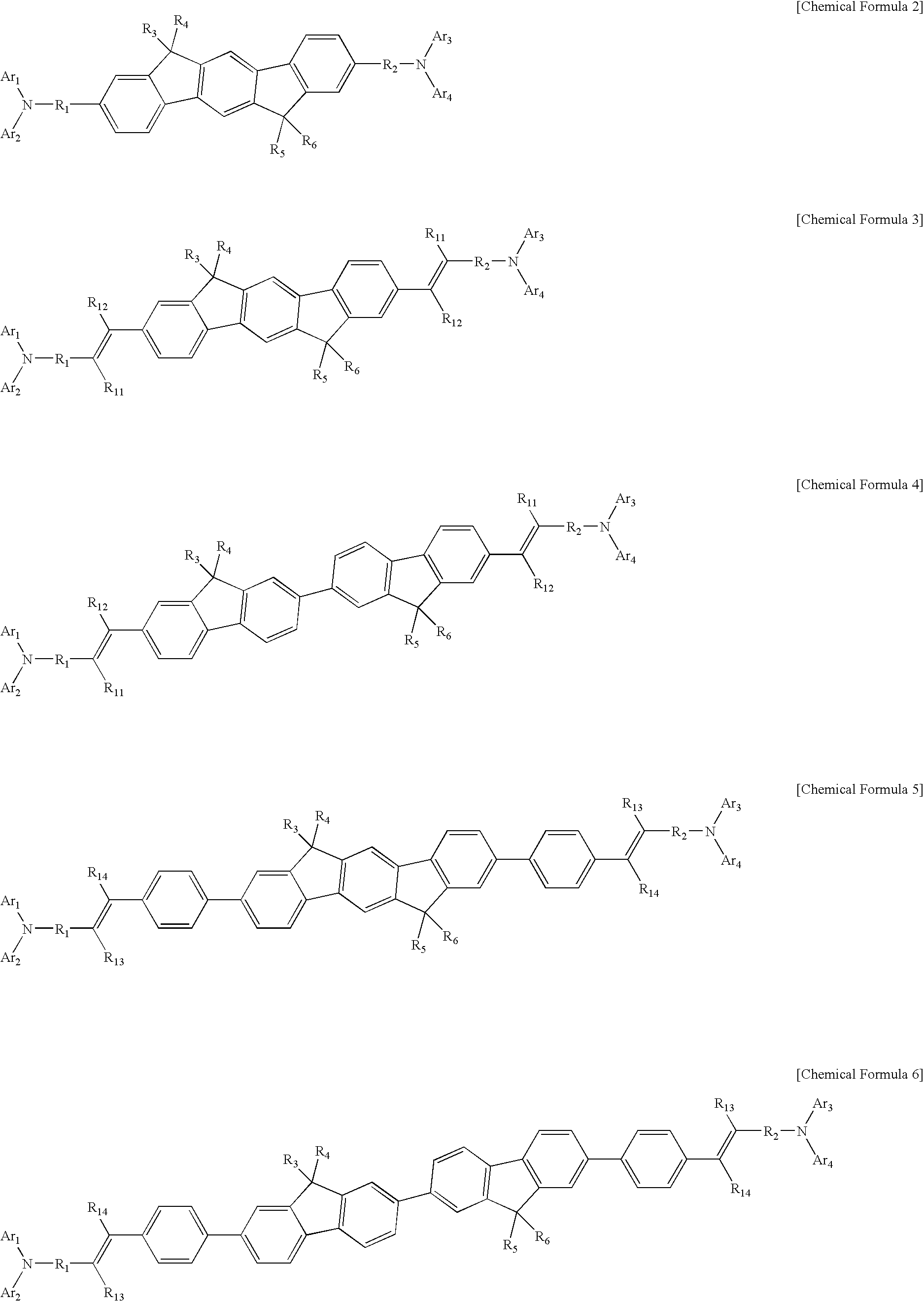

In the compounds represented by Chemical Formula 2 through Chemical Formula 6, R₁ and R₂ independently represent an aromatic group or a fused multi-cyclic aromatic ring having two or more aromatic rings, R₃ through R₆ independently represent a linear or branched C₁-C₂₀ alkyl group with or without halogen substituent(s), a C₅-C₈ cycloalkyl group, or an aromatic group with or without halogen substituent(s), R₁₁ through R₁₄ independently represent a hydrogen, a C₁-C₈ alkyl group, a C₅-C₈ cycloalkyl group or an aromatic group with or without halogen substituent(s), and Ar₁ through Ar₄ independently represent an aromatic ring or a fused multi-cyclic aromatic ring having two or more aromatic rings.

Specific examples of R₁ and R₂ in Chemical Formulas 2 through 6 include phenylene, naphthalene, anthracene, naphthacene, pyrene, fluorene and biphenyl, R₃ through R₆ are methyl, ethyl, n-propyl, i-propyl, n-butyl, i-butyl, t-butyl, n-pentyl, i-amyl, n-hexyl, n-heptyl, n-octyl, 2-ethylhexyl, n-nonyl, trifluoromethyl, pentafluoroethyl, cyclopentyl, cyclohexyl, cycloheptyl, cyclooctyl, phenyl, tolyl, 1-naphthyl, 2-naphthyl, 2-fluorophenyl or 4-fluorophenyl, R₁₁ through R₁₄ are hydrogen, methyl, ethyl, n-propyl, i-propyl, n-butyl, i-butyl, t-butyl, n-pentyl, n-hexyl, n-heptyl, n-octyl, cyclopentyl, cyclohexyl, cycloheptyl, cyclooctyl, phenyl, tolyl, 1-naphthyl, 2-naphthyl, 2-fluorophenyl or 4-fluorophenyl, and Ar₁ through Ar₄ are phenyl, tolyl, xylyl, pyridyl, biphenyl, naphthyl, anthryl, phenanthryl, naphthacenyl, acenaphthenyl, pyrenyl, fluorenyl or perylenyl. The organic electroluminescent compounds according to each one of Chemical Formulas 2 to 6 can be specifically exemplified as the compounds having the structures as follows;

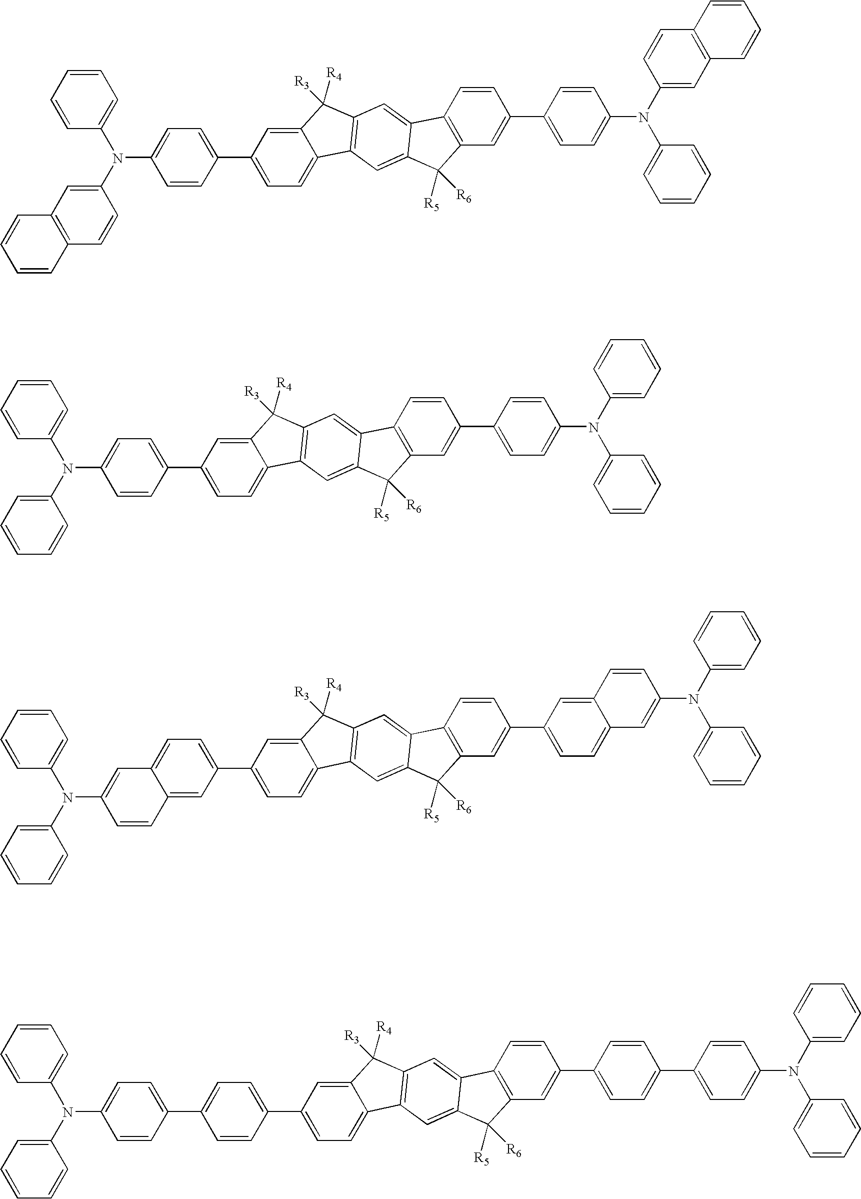

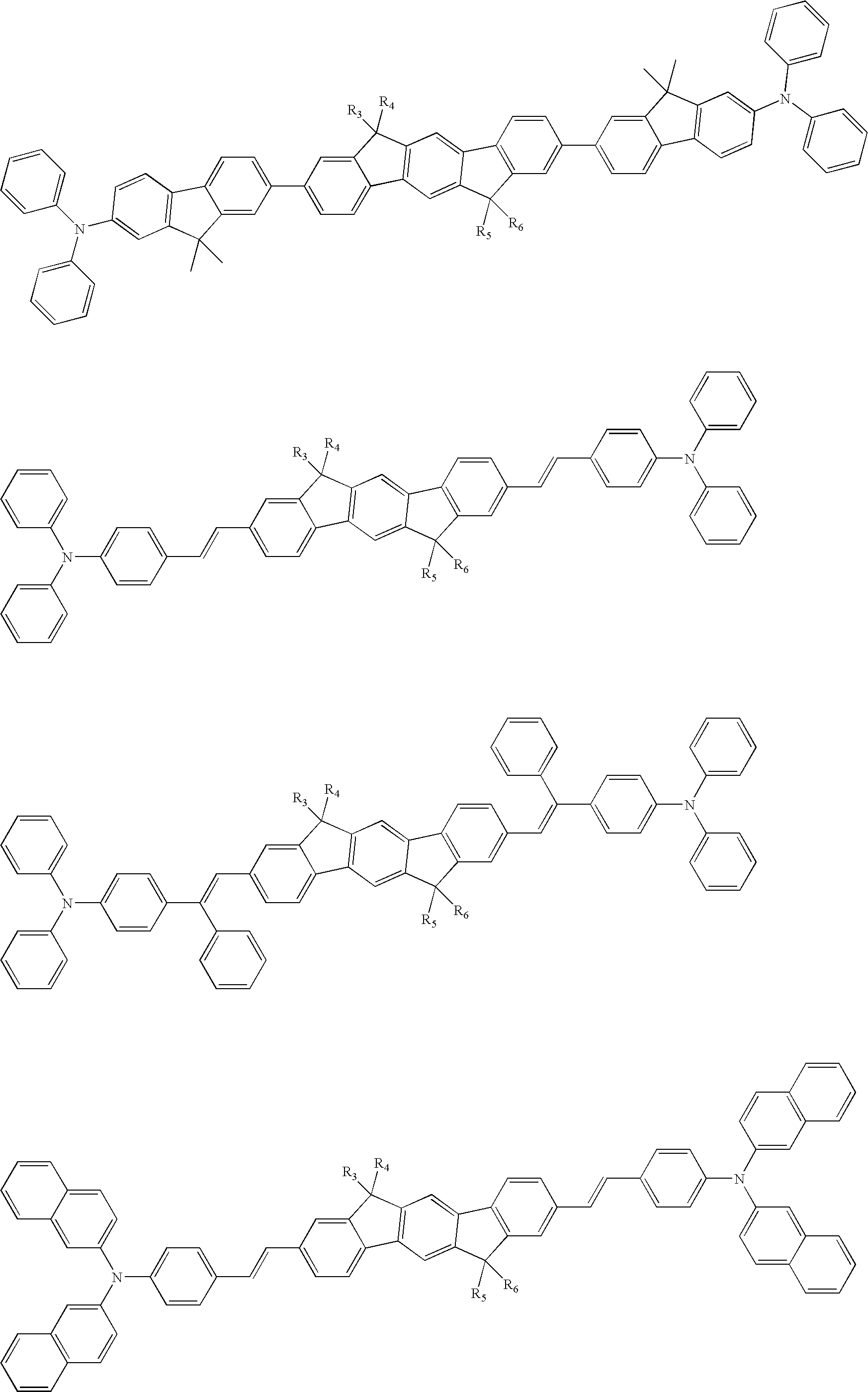

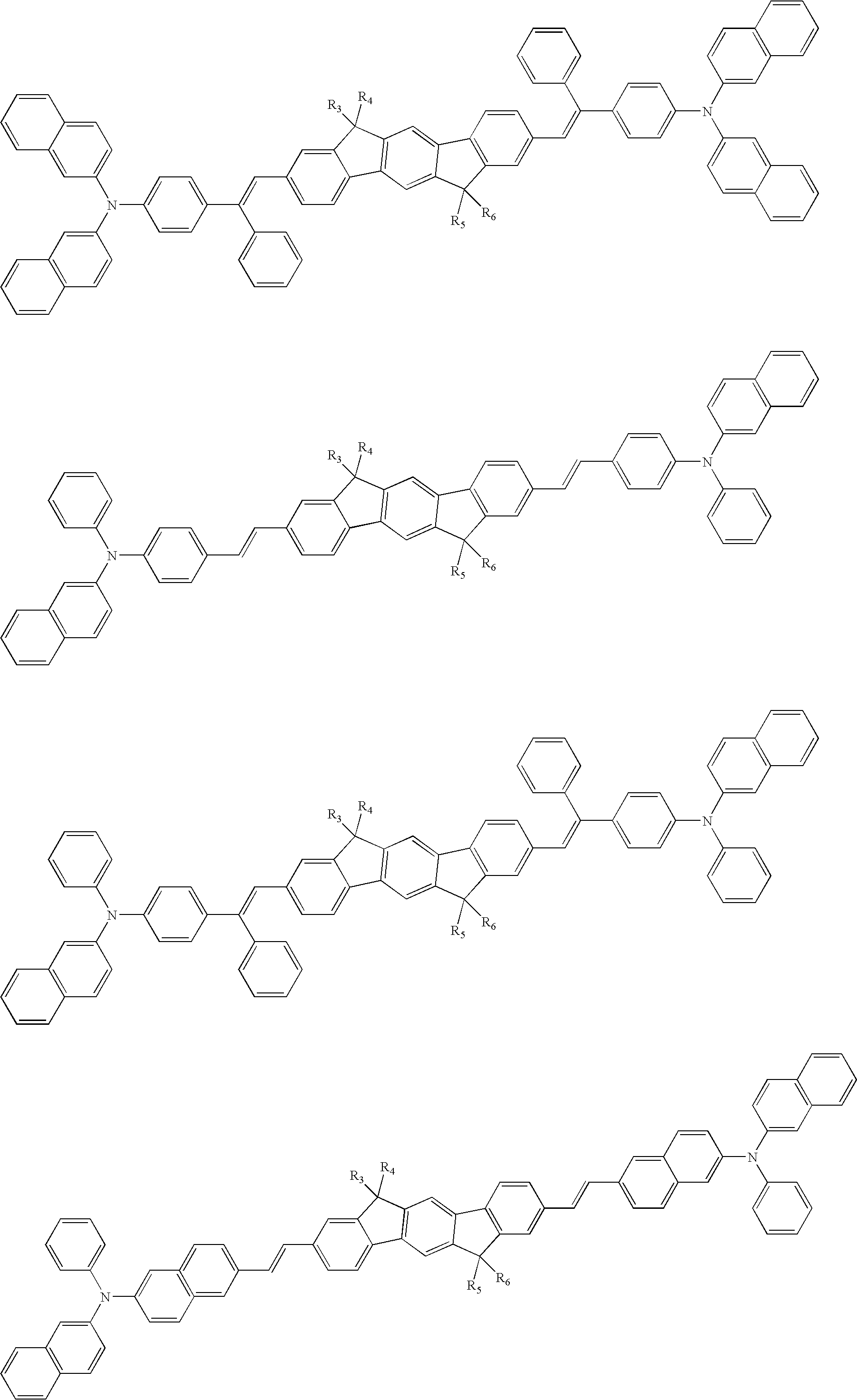

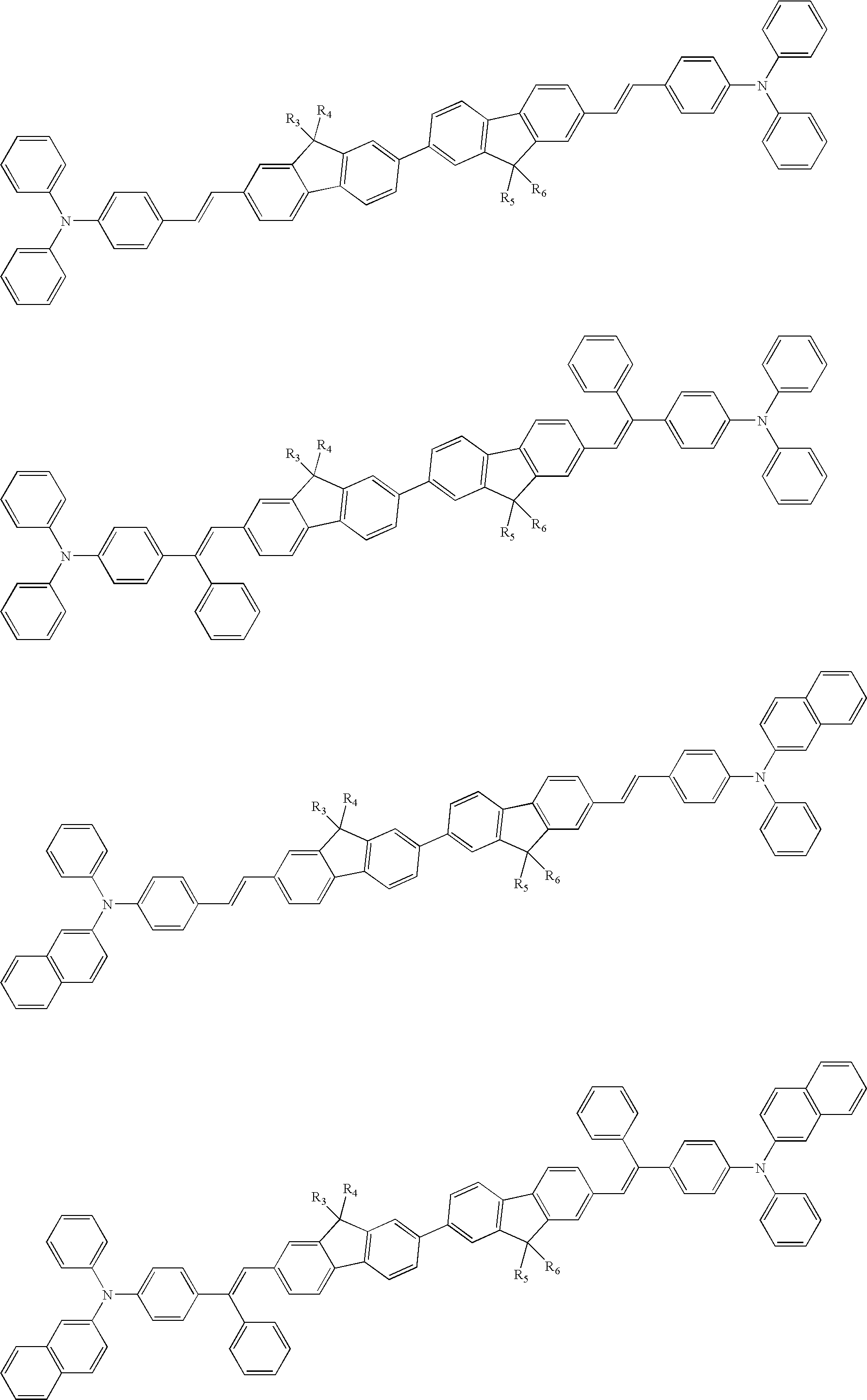

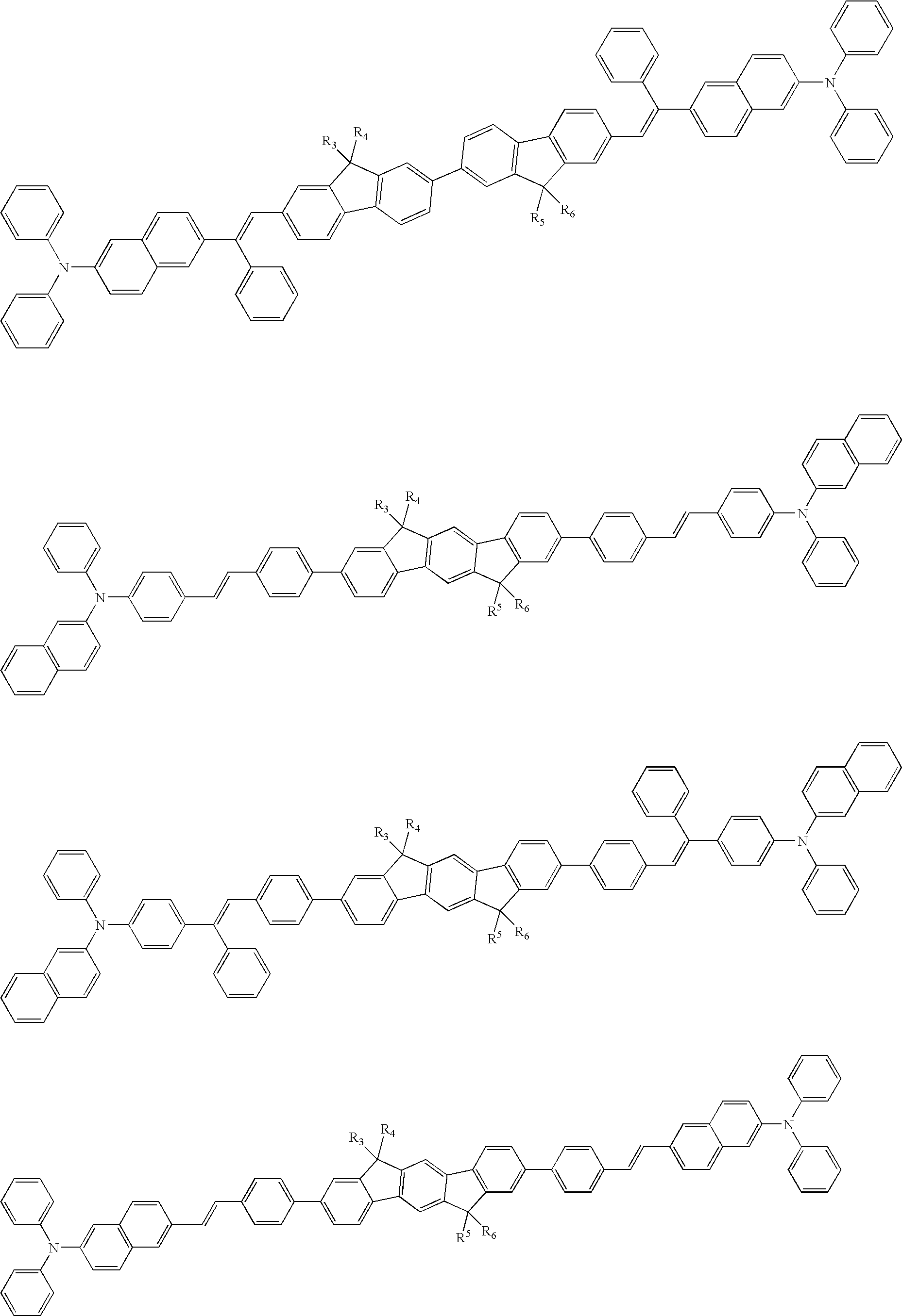

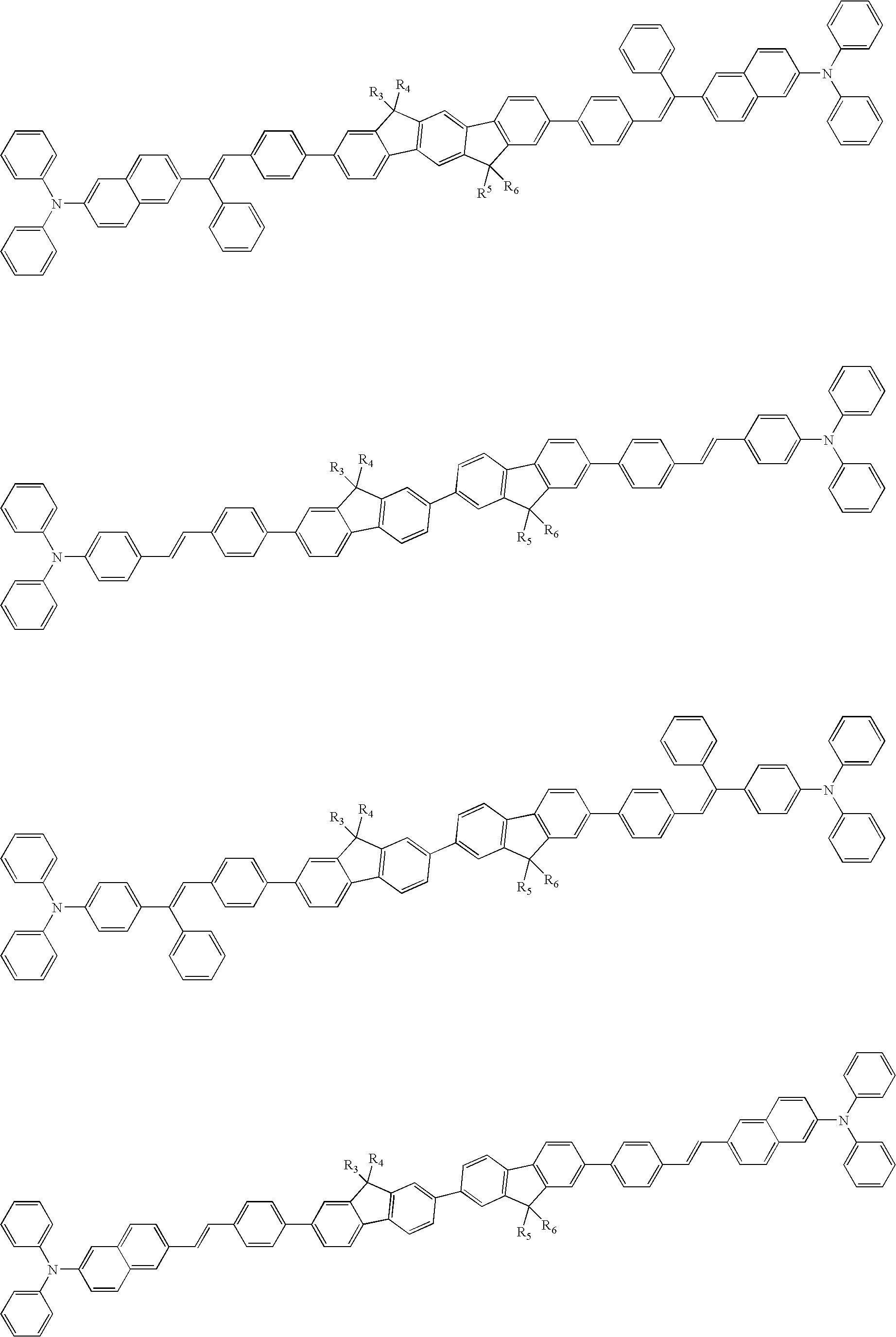

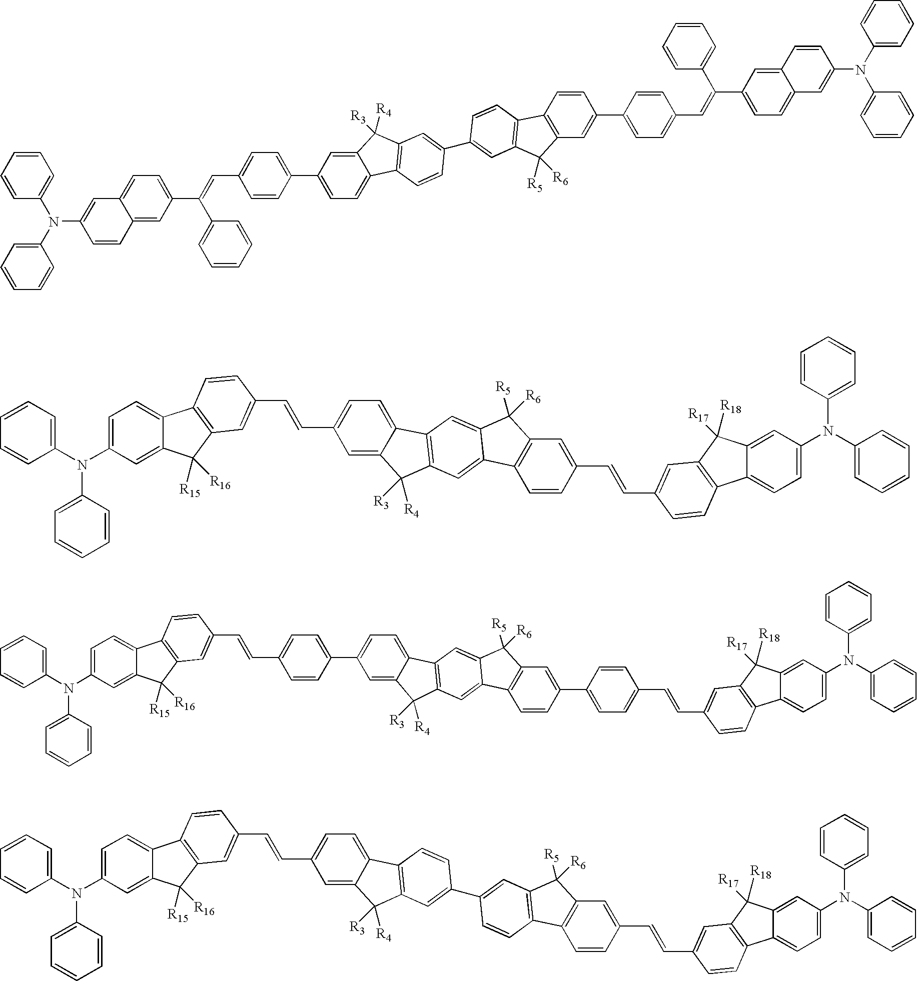

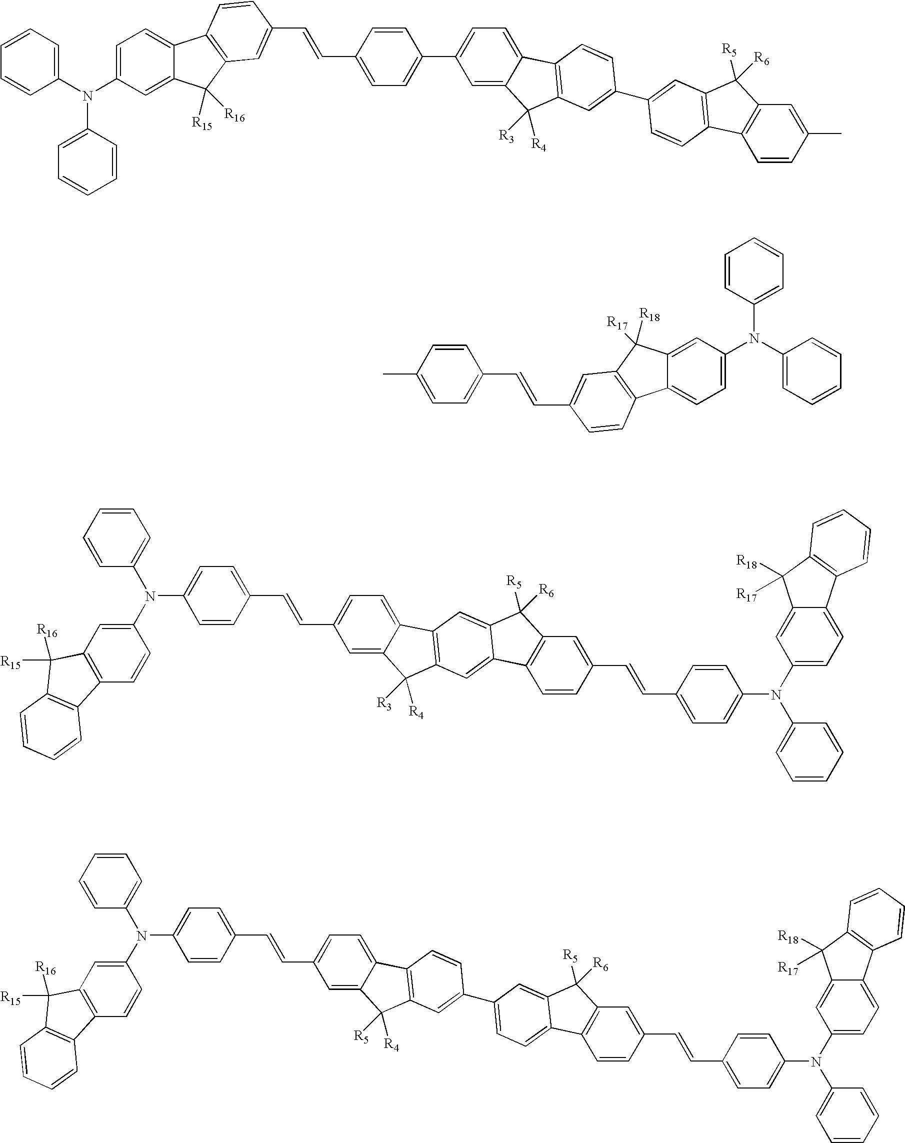

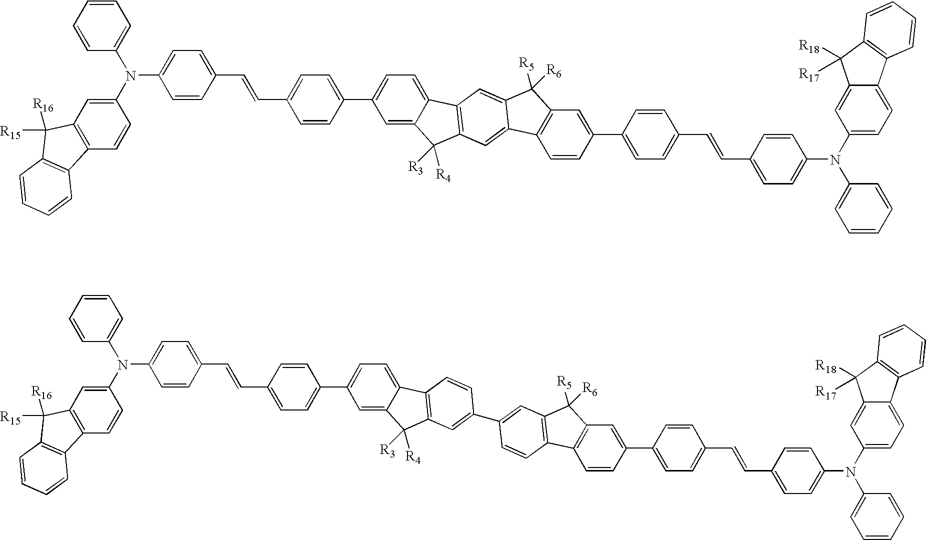

wherein, R₃ through R₆ represent methyl group or ethyl group, and R₁₅ to R₁₈ represent methyl group, ethyl group or phenyl group.

Each compound represented by Chemical Formulas 2, 3 or 5, as an organic electroluminescent compound can be prepared via a reaction route represented by Reaction Scheme 1, a compound of Chemical Formula 4 via a reaction route of Reaction Scheme 2, and a compound of Chemical Formula 6 via that of Reaction Scheme 3.

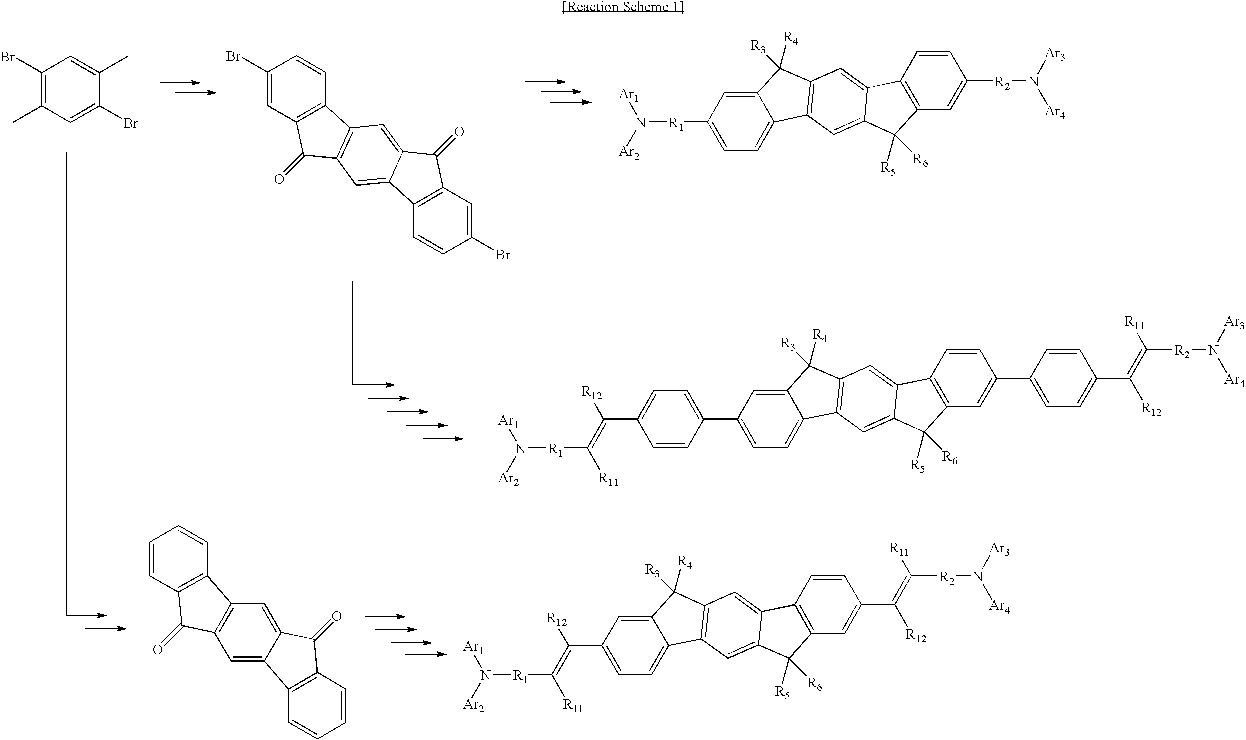

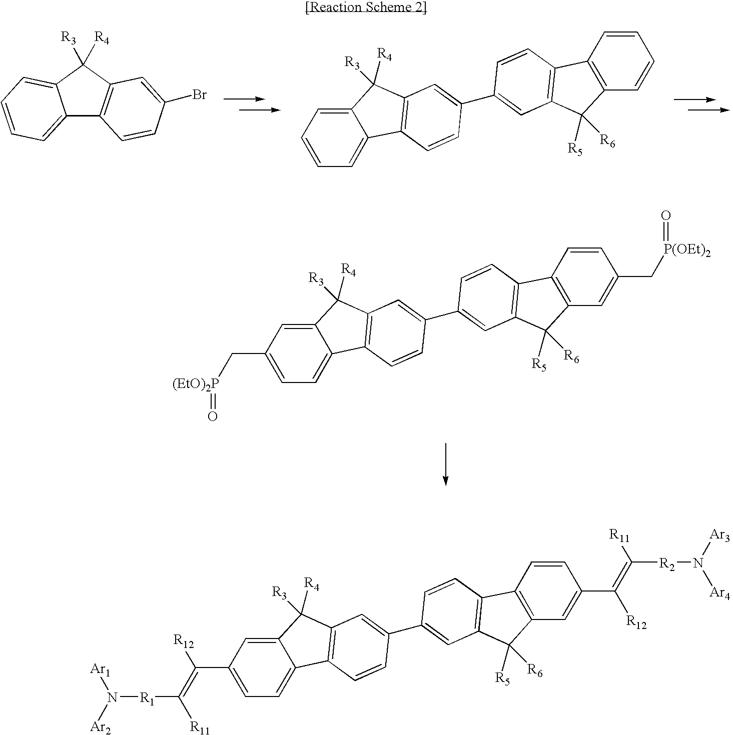

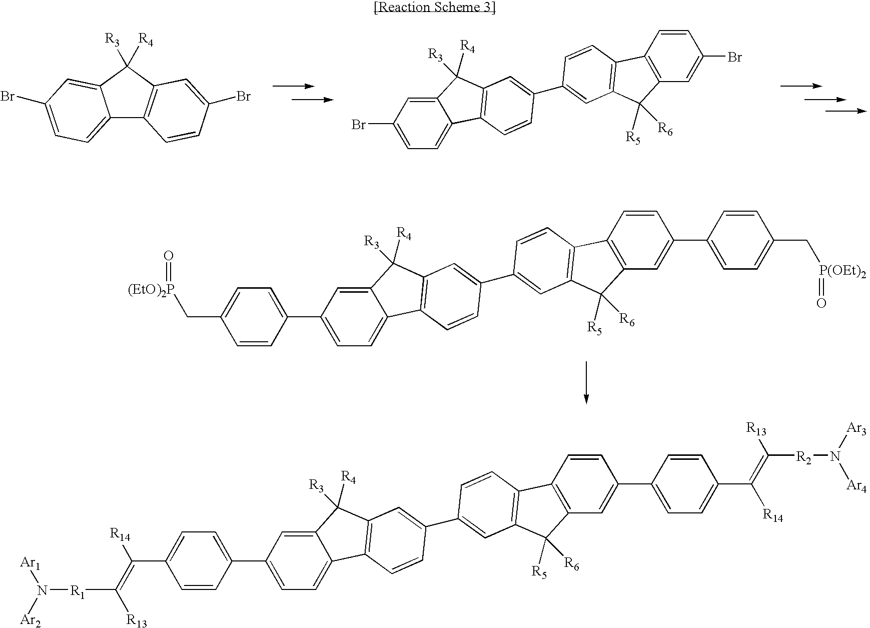

### BRIEF DESCRIPTION OF DRAWINGS

FIG. 1 is a graph showing the EL spectrum of an OLED employing IF2-1 as an electroluminescent material of the present invention and that of Comparative Example 1,

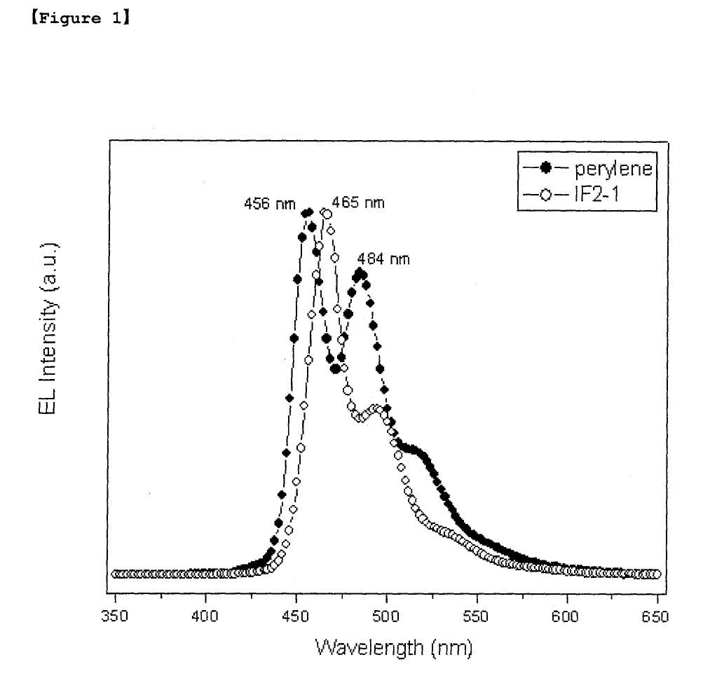

FIG. 2 is a graph showing the change of current density with respect to voltage of an OLED employing IF2-1 as an electroluminescent material of the present invention,

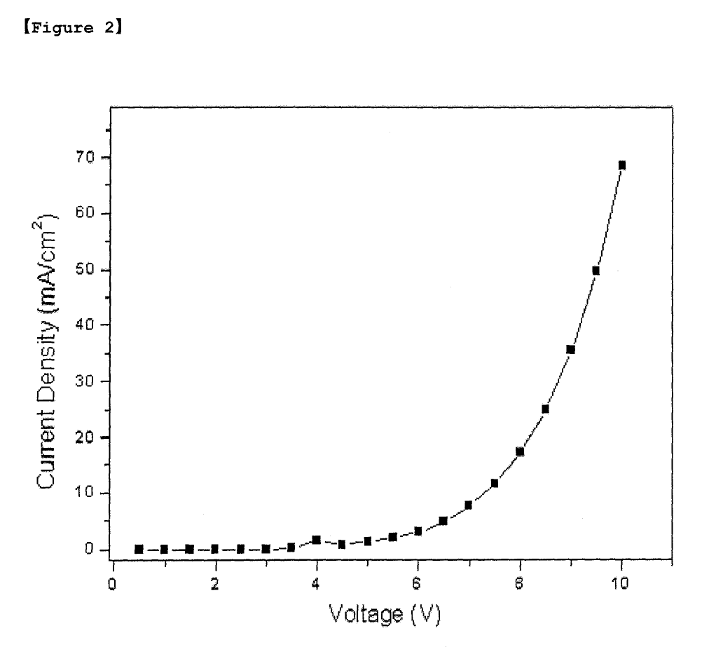

FIG. 3 is a graph showing the change of luminance with respect to the operating voltage of an OLED employing IF2-1 as an electroluminescent material of the present invention, and

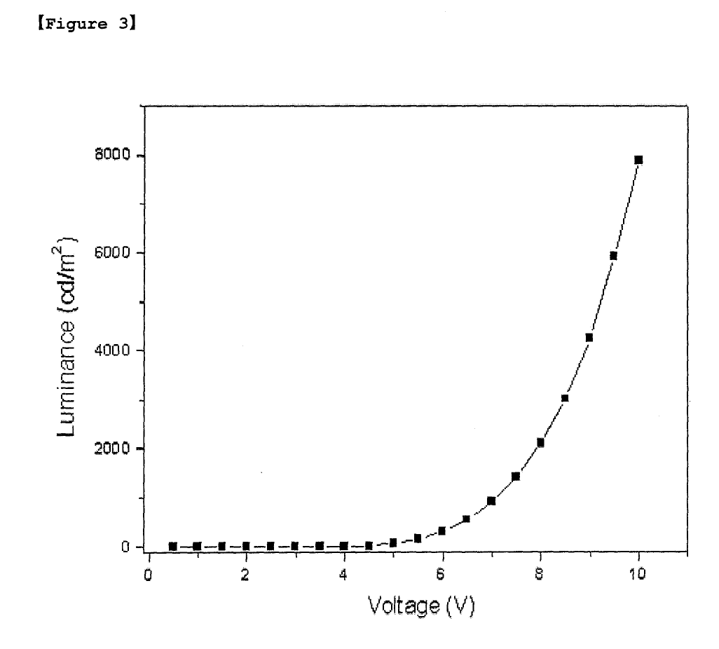

FIG. 4 is a graph showing the change of electroluminescent efficiency with respect to current density of an OLED employing IF2-1 element as an electroluminescent material of the present invention.

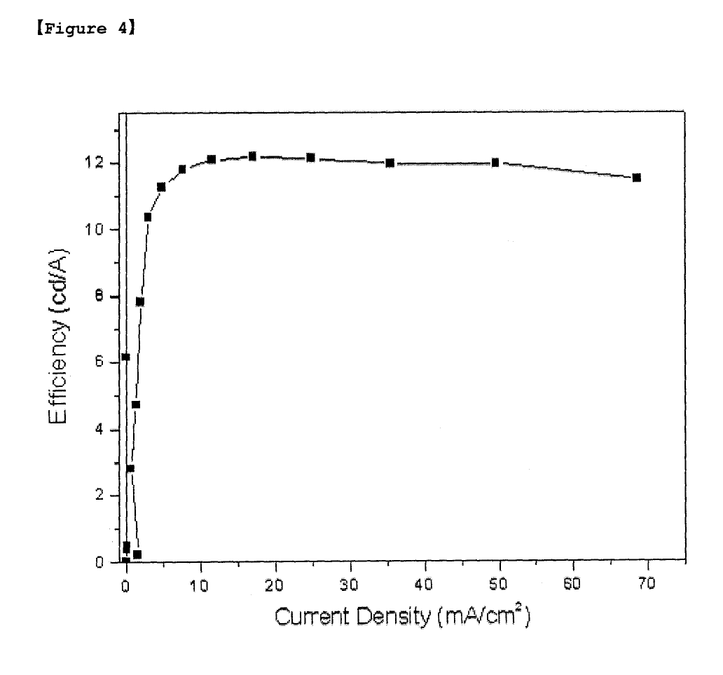

Other and further objects, features and advantages of the invention will appear more fully from the following description.

### EXAMPLES

The present invention is further described with respect to the electroluminescent compounds according to the invention, a process for preparing the same and the electroluminescent properties of the device employing the same by referring to representative compounds according to the present invention, which are provided for illustration only and are not intended to be limiting in any way.

### Synthetic Example 1

Synthesis of IF1-1

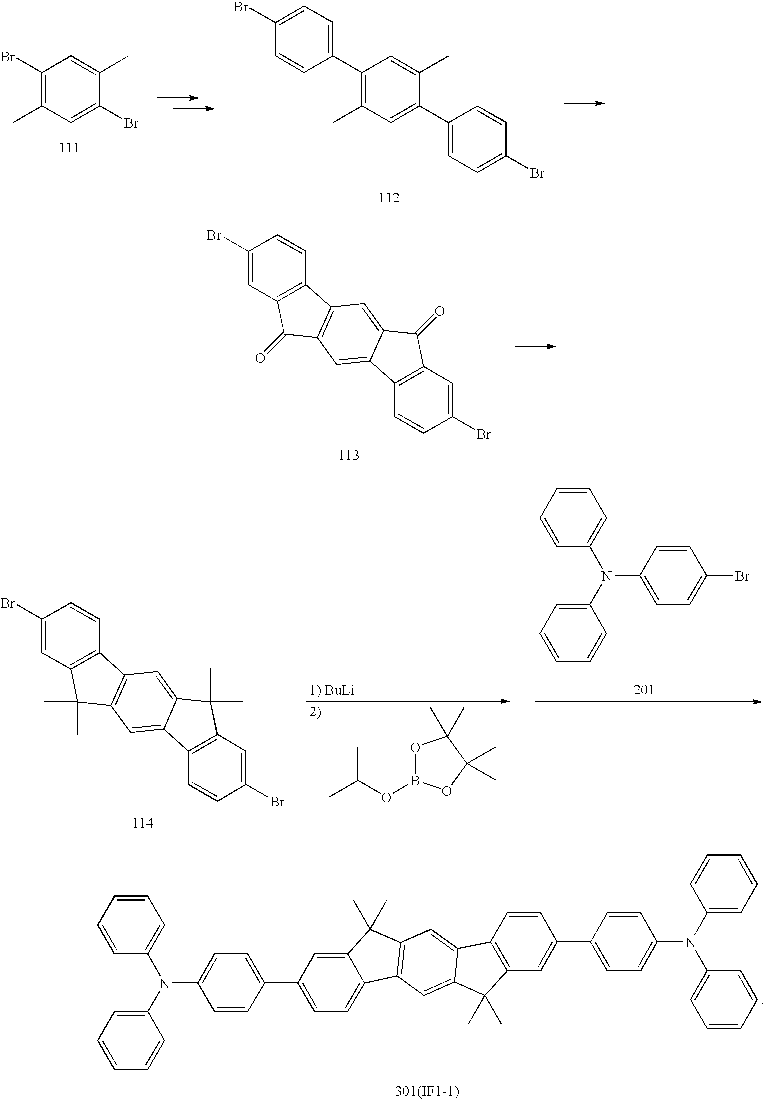

In 50 ml of THF, 2,5-dibromoxylene (2.0 g, 7.60 mmol) as Compound (111) was dissolved and tert-butyl lithium (1.5 equivalent) was added to the THF solution of 2,5-dibromoxylene (50 ml) at −80° C. and the resultant solution was stirred at −40° C. under nitrogen atmosphere for 12 hours. After the stirring completed, 2-isopropoxy-4,4,5,5-tetramethyl-1,3,2-dioxaborolane (4.3 g, 23.1 mmol) was added thereto, while maintaining the temperature of the reaction mixture at −80° C. to provide a boronic ester compound of Compound (111) (2.31 g, 6.42 mmol), and the resultant boronic ester compound of Compound (111) (2.3 g) was dissolved in 50 ml of THF. To the solution, added were 1-bromo-4-iodobenzene (3.80 g, 13.4 mmol), Pd(PPh₃)₄ (1.5 g, 1.29 mmol) and aqueous 2M solution of calcium carbonate (20 ml), and the resultant mixture was heated under reflux for 10 hours. From the precipitate thus formed, Compound (112) (2.14 g, 5.14 mmol) was obtained.

Compound (112) (2.14 g) was dissolved in 20 ml of pyridine, and an aqueous 2M potassium permanganate solution (15 ml) was added thereto to provide a carboxylic acid at the end of Compound (112). The solid obtained from extraction of the organic layer was put into sulfuric acid, and the mixture was heated at 80° C. for 12 hours. After lowering the temperature of said sulfuric acid solution to ambient temperature, the solution was poured into crushed ice. The solid thus formed was extracted to obtain Compound (113) (1.70 g, 3.86 mmol).

Compound (113) (1.70 g) was put into diethylene glycol (30 ml), and hydrazine hydrate (0.58 g, 11.6 mmol) and potassium hydroxide (0.5. g) were added thereto, and the resultant mixture was heated under reflux for 48 hours. After completion of heating under reflux, the reaction mixture was cooled to ambient temperature and extracted from ethyl acetate to provide indinofluorene compound. After drying, the compound was dissolved in 30 ml of THF, and under nitrogen atmosphere, methyl iodide (2.20 g, 15.5 mmol) was added thereto at −78° C. and then 1.8 M solution of n-butyllithium in THF (15 ml) was slowly added thereto. After stirring the mixture for 1 hour, the temperature was raised to ambient temperature, and the mixture was again stirred and the reaction quenched by slowly adding 50 ml of water. After completion of the reaction, the reaction mixture was extracted from the organic layer and completely dried to obtain Compound (114) (1.17 g, 2.50 mmol).

Compound (114) (1.17 g) was dissolved in THF (30 ml), and 1.5 equivalent of tert-butyl lithium was added thereto. By the use of 2-isopropoxy-4,4,5,5-tetramethyl-1,3,2-dioxaborolane (1.4 g, 7.53 mmol), a boronic ester compound (1.33 g, 2.37 mmol) as Compound (114) was prepared. The boronic ester compound of Compound (114) (1.33 g) thus obtained was dissolved in 30 ml of THF solution, and Compound (201) (1.56 g, 4.81 mmol) and Pd(PPh₃)₄ (0.56 g, 0.48 mmol) and aqueous 2M solution of calcium carbonate (10 ml) were added thereto. After heating the reaction mixture under reflux for 12 hour, the precipitate thus formed was extracted with ethyl acetate. After recrystallization and drying, Compound (301, IF1-1) (1.50 g, 1.88 mmol) was obtained as the title compound in total yield of 24.7%.

¹H NMR(200 MHz, CDCl₃): δ 1.65(s, 12H), 6.45-6.55(m, 12H), 6.6-6.65(m, 4H), 7.0-7.05(m, 8H), 7.2-7.25(d, 4H), 7.65(d, 2H), 7.73(d, 2H), 7.80(s, 2H), 8.0-8.1(d, 2H) MS/FAB: 796(found), 797.06(calculated)

### Synthetic Example 2

Synthesis of IF1-2.

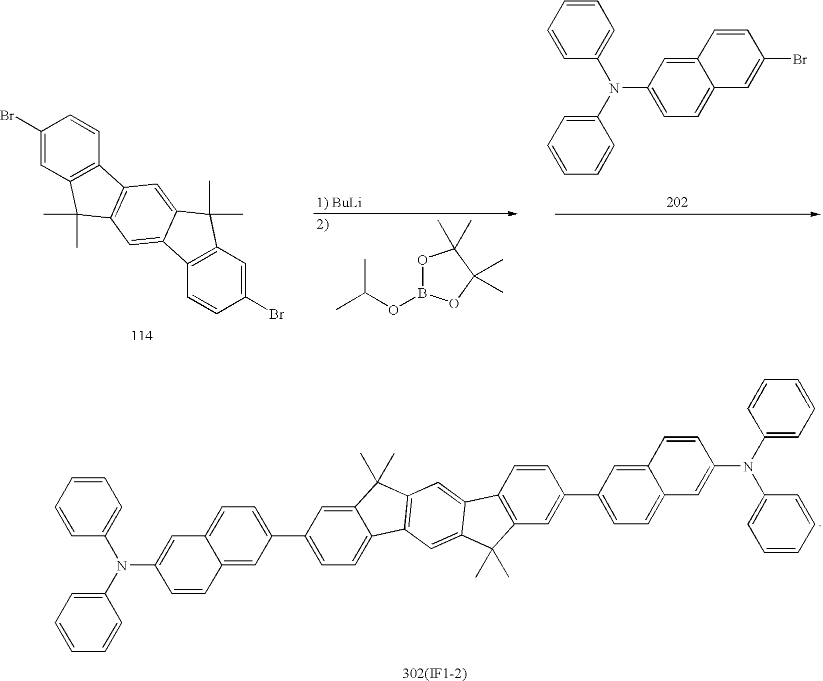

Compound (302, IF1-2) (1.16 g, 1.30 mmol) as the title compound was obtained in total yield of 19.0% according to the same procedure as Synthetic Example 1, but Compound 114 (1.0 g, 2.14 mmol), and Compound 202 (1.61 g, 4.30 mmol) instead of Compound 201 were employed.

¹H NMR(200 MHz, CDCl₃): δ 1.65(s, 12H), 6.45-6.5(m, 8H), 6.6-6.65(m, 4H), 6.75-6.8(m, 4H), 7.0-7.05(m, 8H), 7.45-7.55(m, 6H), 7.65(d, 2H), 7.75-7.85(m, 6H), 8.0-8.05(d, 2H) MS/FAB: 896(found), 897.19(calculated)

### Synthetic Example 3

Synthesis of IF1-3

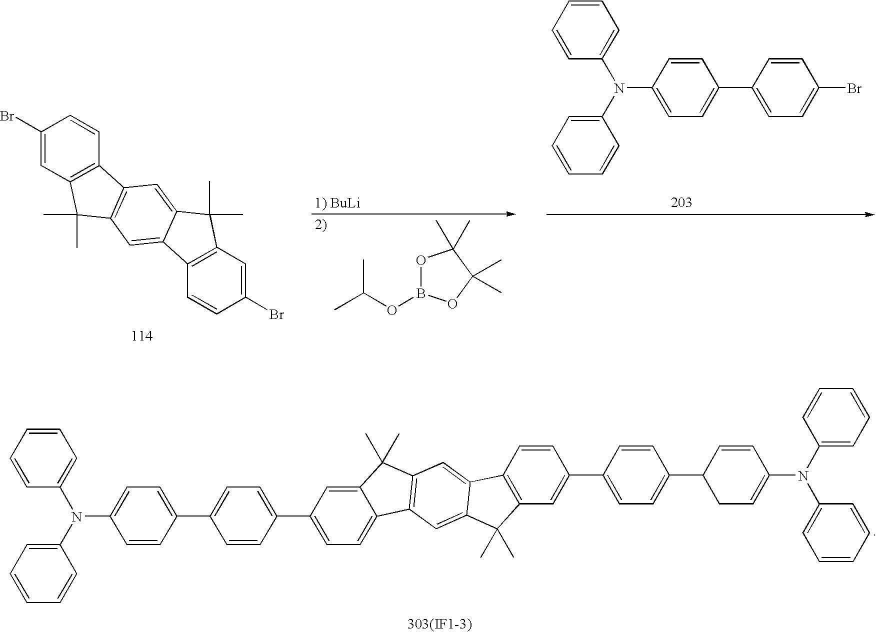

Compound (303, IF1-3) (1.12 g, 1.18 mmol) as the title compound was obtained in total yield of 17.2% according to the same procedure as Synthetic Example 1, but Compound 114 (1.0 g, 2.14 mmol), and Compound 203 (1.72 g, 4.30 mmol) instead of Compound 201 were employed.

¹H NMR(200 MHz, CDCl₃): δ 1.65(s, 12H), 6.45-6.55(m, 12H), 6.6-6.65(m, 4H), 7.0-7.05(m, 8H), 7.2-7.25(d, 4H), 7.55-7.57(d, 8H), 7.65(d, 2H), 7.75(s, 2H), 7.8(s, 2H), 8.0-8.05(d, 2H) MS/FAB: 948(found), 949.26(calculated)

### Synthetic Example 4

Synthesis of IF1-4

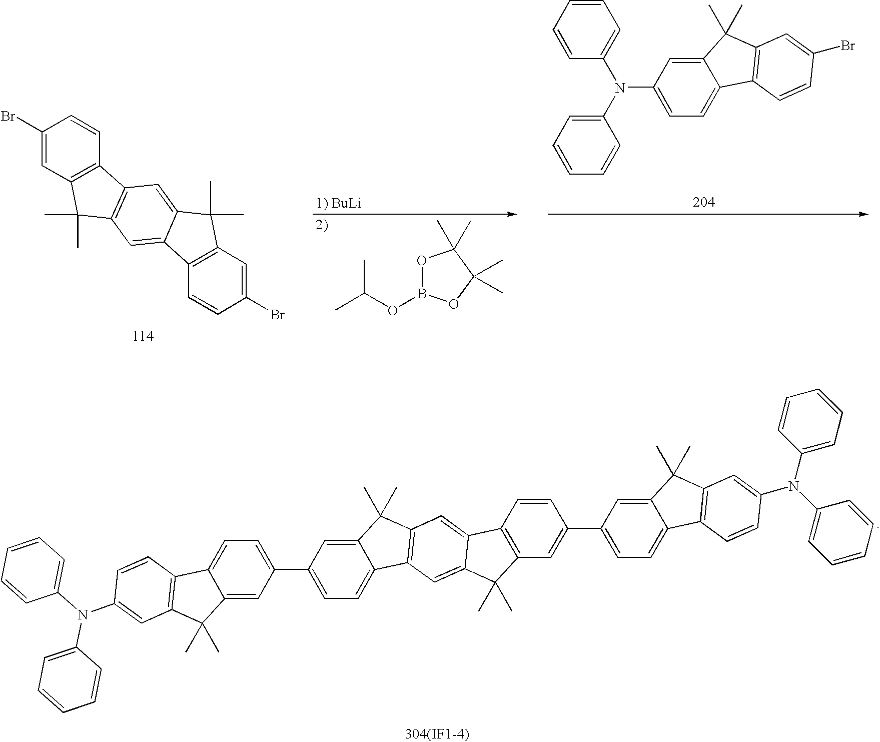

Compound (304, IF1-4) (0.95 g, 0.92 mmol) as the title compound was obtained in total yield of 13.4% according to the same procedure as Synthetic Example 1, but Compound 114 (1.0 g, 2.14 mmol), and Compound 204 (1.90 g, 4.32 mmol) instead of Compound 201 were employed.

¹H NMR(200 MHz, CDCl₃): δ 1.63-1.65(d, 24H), 6.45-6.50(d, 8H), 6.55-6.65(m, 6H), 6.75(s, 2H), 7.0-7.05(m, 8H), 7.58-7.65(m, 6H), 7.73-7.85(m, 6H), 8.0-8.05(d, 2H) MS/FAB: 1028(found), 1029.39(calculated)

### Synthetic Example 5

Synthesis of IF2-1

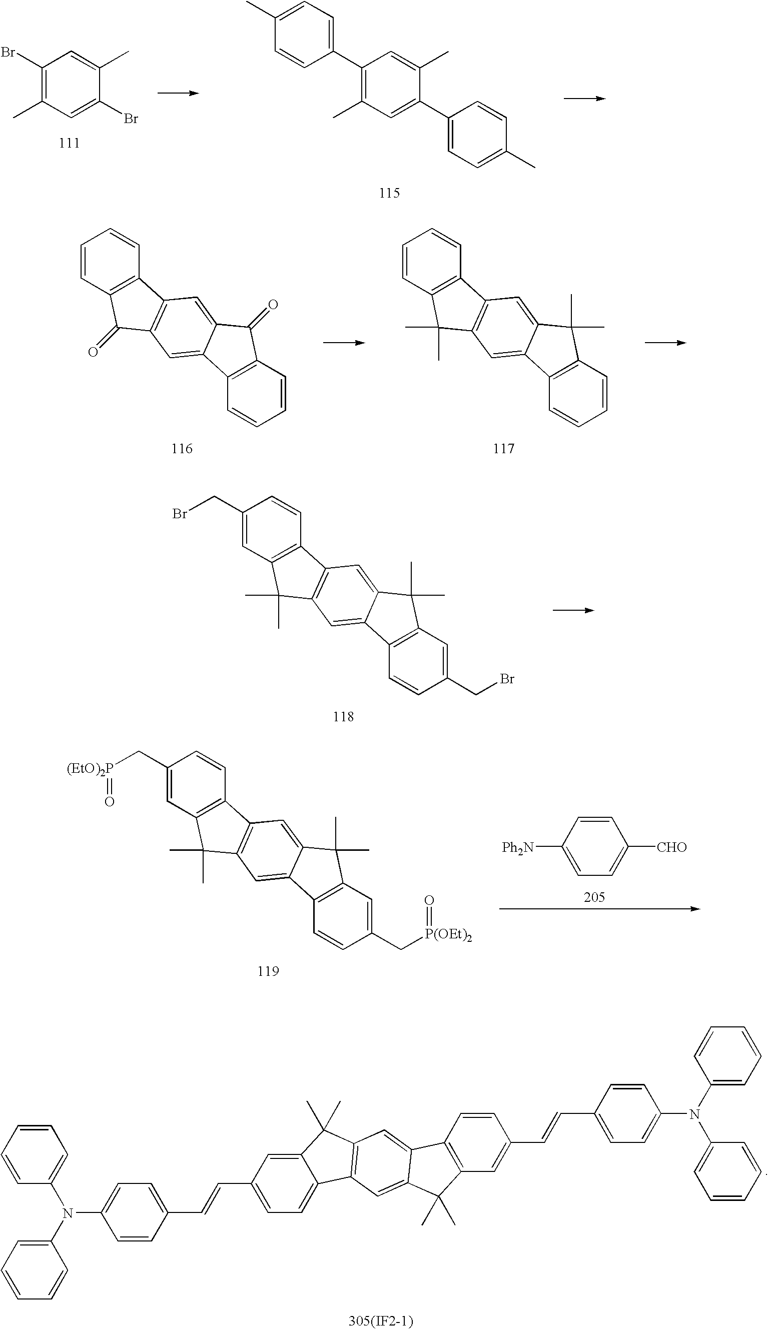

In 50 ml of THF, 2,5-dibromoxylene (2.0 g, 7.60 mmol) was dissolved, and phenyl boronic acid (1.95 g, 16.0 mmol) and Pd(PPh₃)₄ (1.95 g, 1.68 mmol), aqueous 2M calcium carbonate solution (25 ml) were added to the THF solution of 2,5-dibromoxylene (50 ml), and the resultant mixture was heated under reflux for 10 hours. From the precipitate thus formed, Compound (115) (1.86 g, 7.2 mmol) was obtained.

Compound (115) (1.86 g) was dissolved in 20 ml of pyridine, and an aqueous 2M potassium permanganate solution (15 ml) was added thereto to provide carboxylic acid at the end of the compound. The solid obtained from extraction of the organic layer was put into sulfuric acid, and the mixture was heated at 80° C. for 12 hours. After lowering the temperature of said sulfuric acid solution to ambient temperature, the solution was poured into crushed ice. The solid thus formed was extracted to obtain Compound (116) (1.64 g, 5.82 mmol).

Compound (116) (1.64 g) was put into diethylene glycol (30 ml), and hydrazine hydrate (0.85 g, 17.0 mmol) and potassium hydroxide (0.8 g) were added thereto, and the resultant mixture was heated under reflux for 48 hours. After completion of heating under reflux, the reaction mixture was cooled to ambient temperature and extracted from ethyl acetate to provide indinofluorene compound. After drying, the compound was dissolved in 30 ml of THF, and under nitrogen atmosphere, methyl iodide (1.65 g, 11.6 mmol) was added thereto at −78° C. and then 1.8 M solution of n-butyllithium in THF (12 ml) was slowly added thereto. After stirring the mixture for 1 hour, the temperature was raised to ambient temperature, and the mixture was again stirred for 24 hours and the reaction was quenched by slowly adding 50 ml of water. After completion of the reaction, the reaction mixture was extracted from organic layer and completely dried to obtain Compound (117) (1.25 g, 4.03 mmol).

Compound (117) (1.25 g, 4.03 mmol) and paraformaldehyde (1.8 g) were added to 35% HBr solution in acetic acid (15 ml), and the mixture was heated at 60° C. for 24 hours. The temperature of the reaction mixture was then lowered to ambient temperature, extracted and dried to obtain Compound (118) (1.34 g, 2.70 mmol), which was charged in a reaction vessel. Triethylphosphite (2.0 g, 12.0 mmol) was added thereto at 0° C. and the mixture was heated at 150° C. for 4 hours. After completion of the reaction, residual triethylphosphite was removed by vacuum distillation, and the residue was extracted from ethyl acetate to obtain Compound (119) (1.4 g, 2.30 mmol) Compound (119) (1.4 g) and Compound (205) (1.30 g, 4.76 mmol) were dissolved in THF (30 ml), and 1.6 M solution of potassium tert-butoxide in THF (5 ml) was added dropwise to the mixed solution. The temperature was slowly raised to ambient temperature to complete the reaction. Then an excess amount of water was poured thereto to generate solid, which was then filtered. After recrystallization from THF-methanol, Compound (305, IF2-1) (1.23 g, 1.45 mmol) was obtained as the title compound in total yield of 19.1%.

¹H NMR(200 MHz, CDCl₃): δ 1.65(s, 12H), 6.45-6.5(m, 12H), 6.6-6.65(m, 4H), 6.95-7.05(m, 12H), 7.15-7.2(d, 4H), 7.57-7.6(d, 2H), 7.7-7.75(d, 4H), 7.9-8.0(d, 2H) MS/FAB: 848(found), 849.14(calculated)

### Synthetic Example 6

Synthesis of IF2-2

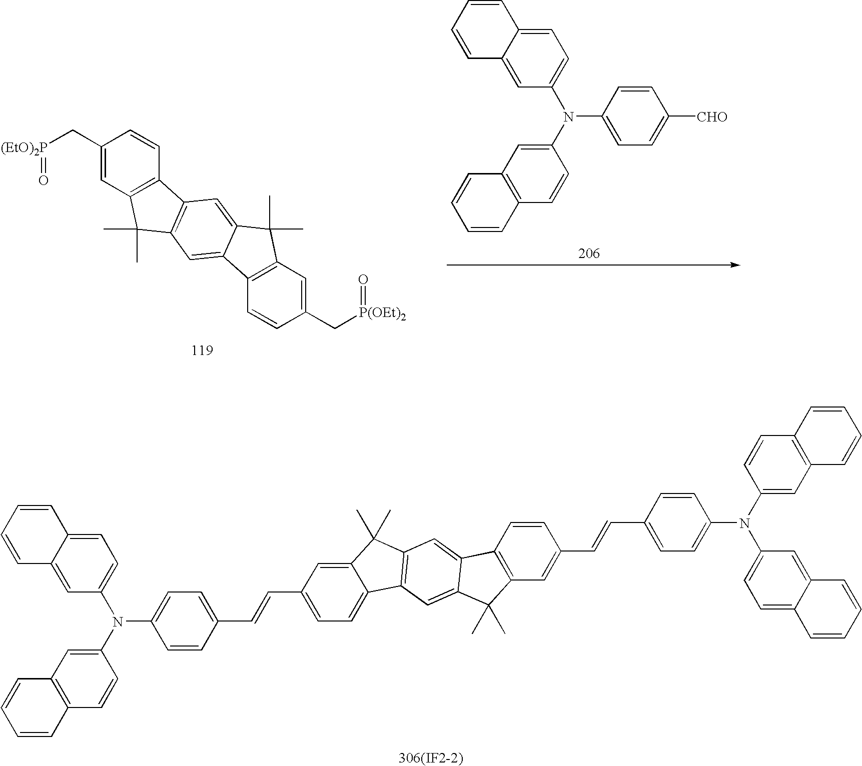

Compound (306, IF2-2) (0.95 g, 0.91 mmol) as the title compound was obtained in total yield of 16.8% according to the same procedure as Synthetic Example 5, but Compound 119 (1.0 g, 1.64 mmol), and Compound 206 (1.28 g, 3.43 mmol) instead of Compound 205 were employed.

¹H NMR(200 MHz, CDCl₃): δ 1.65(s, 12H), 6.45-6.50(d, 4H), 6.75-6.8(m, 8H), 7.0(d, 4H), 7.15-7.3(m, 12H), 7.4-7.6(m, 14H), 7.7-7.75(d, 4H), 7.9-8.0(d, 2H) MS/FAB: 1048(found), 1049.38(calculated)

### Synthetic Example 7

Synthesis of IF2-3

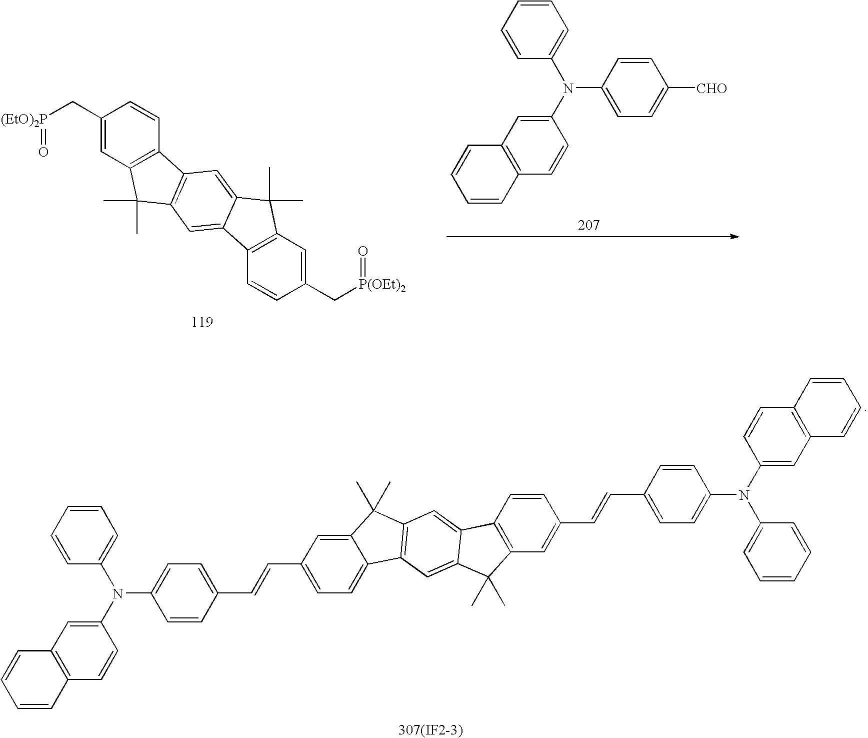

Compound (307, IF2-3) (0.92 g, 1.08 mmol) as the title compound was obtained in total yield of 20.0% according to the same procedure as Synthetic Example 5, but Compound 119 (1.0 g, 1.64 mmol), and Compound 207 (1.10 g, 3.41 mmol) instead of Compound 205 were employed.

¹H NMR(200 MHz, CDCl₃): δ 1.65(s, 12H), 6.45-6.5(d, 8H), 6.6-6.65(m, 2H), 6.75-6.8(m, 4H), 6.95-7.25(m, 16H), 7.4-7.6(m, 8H), 7.7-7.75(d, 4H), 7.9-8.0(d, 2H) MS/FAB: 948(found), 949.26(calculated)

### Synthetic Example 8

Synthesis of IF2-4

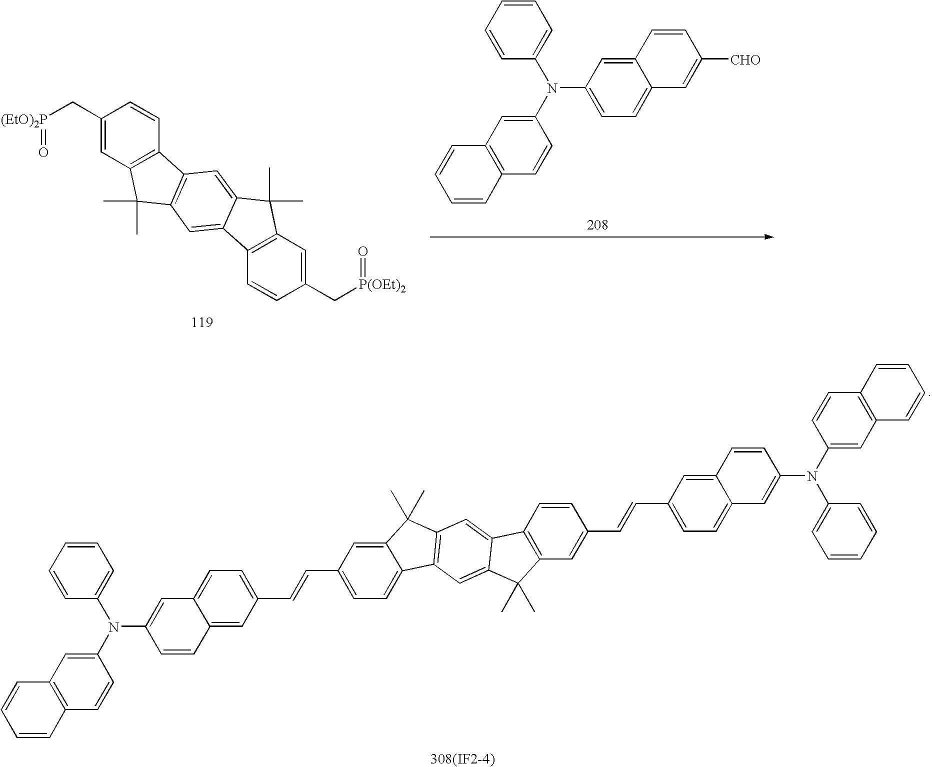

Compound (308, IF2-4) (0.71 g, 0.68 mmol) as the title compound was obtained in total yield of 12.6% according to the same procedure as Synthetic Example 5, but Compound 119 (1.0 g, 1.64 mmol), and Compound 208 (1.28 g, 3.43 mmol) instead of Compound 205 were employed.

¹H NMR(200 MHz, CDCl₃): δ 1.65(s, 12H), 6.45-6.5(d, 4H), 6.6-6.65(m, 2H), 6.75-6.8(m, 8H), 6.95-7.1(m, 10H), 7.2-7.25(m, 2H), 7.35-7.6(m, 14H), 7.7-7.75(d, 6H), 7.9-8.0(d, 2H) MS/FAB: 1048(found), 1049.38(calculated)

### Synthetic Example 9

Synthesis of DF1-1

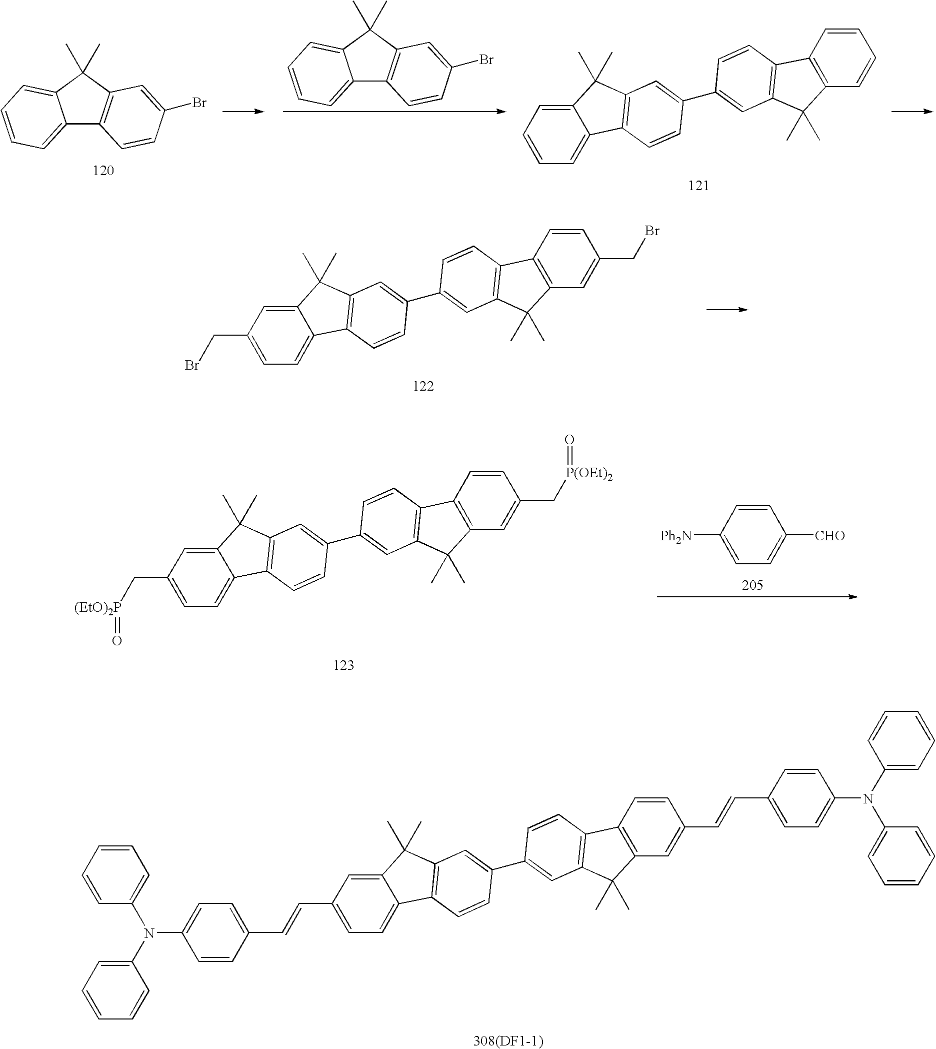

In 50 ml of THF, 2-bromo-9,9-dimethylfluorene (2.0 g, 7.33 mmol) as Compound (120) was dissolved and tert-butyl lithium (1.5 equivalent) was added to the THF solution of 2-bromo-9,9-dimethylfluorene (50 ml) at −80° C. and the resultant solution was stirred at −40° C. under nitrogen atmosphere for 12 hours.

After the stirring completed, 2-isopropoxy-4,4,5,5-tetramethyl-1,3,2-dioxaborolane (4.3 g, 23.1 mmol) was added thereto, while maintaining the temperature of the reaction mixture at −80° C. to provide a boronic ester compound of Compound (120) (2.16 g, 6.75 mmol), and the resultant boronic ester compound of Compound (120) (2.16 g) was dissolved in 50 ml of THF. To the solution, 2-bromo-9,9-dimethylfluorene (1.92 g, 7.03 mmol), Pd(PPh₃)₄ (0.97 g, 0.84 mmol) and aqueous 2M solution of calcium carbonate (15 ml) were added and the resultant mixture was heated under reflux for 10 hours. From the precipitate thus formed, Compound (121) (2.26 g, 5.85 mmol) was obtained.

Compound (121) (2.26 g) and paraformaldehyde (2.6 g) were added to 35% HBr solution in acetic acid (20 ml), and the mixture was heated at 60° C. for 24 hours. Then the temperature of the reaction mixture was lowered to ambient temperature, extracted and dried to obtain Compound (122) (2.64 g, 4.62 mmol). Compound (122) (2.64 g) was charged to a reaction vessel. Triethylphosphite (3.42 g, 20.6 mmol) was added thereto at 0° C. and the mixture was heated at 150° C. for 4 hours. After completion of the reaction, residual triethylphosphite was removed by vacuum distillation, and the residue was extracted from ethyl acetate to obtain Compound (123) (2.13 g, 3.10 mmol).

Compound (123) (2.13 g) and Compound (205) (1.86 g, 6.81 mmol) were dissolved in THF (40 ml), and 1.6 M solution of potassium tert-butoxide in THF (10 ml) was added dropwise to the mixed solution at 0° C. The temperature was slowly raised to ambient temperature to complete the reaction. Then an excess amount of water was poured thereto to generate solid, which was then filtered. After recrystallization form THF-methanol, Compound (309, DF1-1) (1.54 g, 1.67 mmol) was obtained as the title compound in total yield of 22.8%.

¹H NMR(200 MHz, CDCl₃): δ 1.65(s, 12H), 6.45-6.5(m, 12H), 6.6-6.65(m, 4H), 6.95-7.05(m, 12H), 7.15-7.2(d, 4H), 7.5-7.65(m, 4H), 7.7-7.8(m, 4H), 7.85-7.9(d, 4H) MS/FAB: 924(found), 925.24(calculated)

### Synthetic Example 10

Synthesis of DF1-2

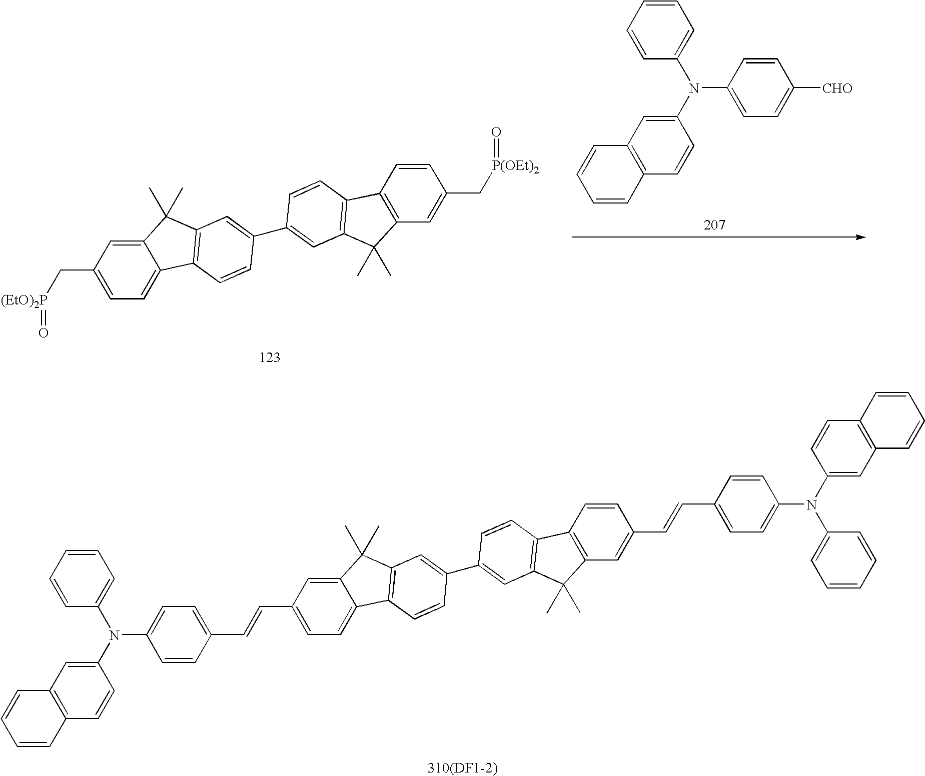

Compound (310, DF1-2) (0.88 g, 0.86 mmol) as the title compound was obtained in total yield of 24.9% according to the same procedure as Synthetic Example 9, but Compound 123 (1 g, 1.46 mmol), and Compound 207 (1.03 g, 3.19 mmol) instead of Compound 205 were employed.

¹H NMR(200 MHz, CDCl₃): δ 1.65(s, 12H), 6.45-6.5(m, 8H), 6.6-6.65(m, 2H), 6.75-6.8(m, 4H), 6.95-7.25(m, 16H), 7.35-7.6(m, 10H), 7.7-7.8(d, 4H), 7.8-7.9(d, 4H) MS/FAB: 1024(found), 1025.36(calculated)

### Synthetic Example 11

Synthesis of DF1-3

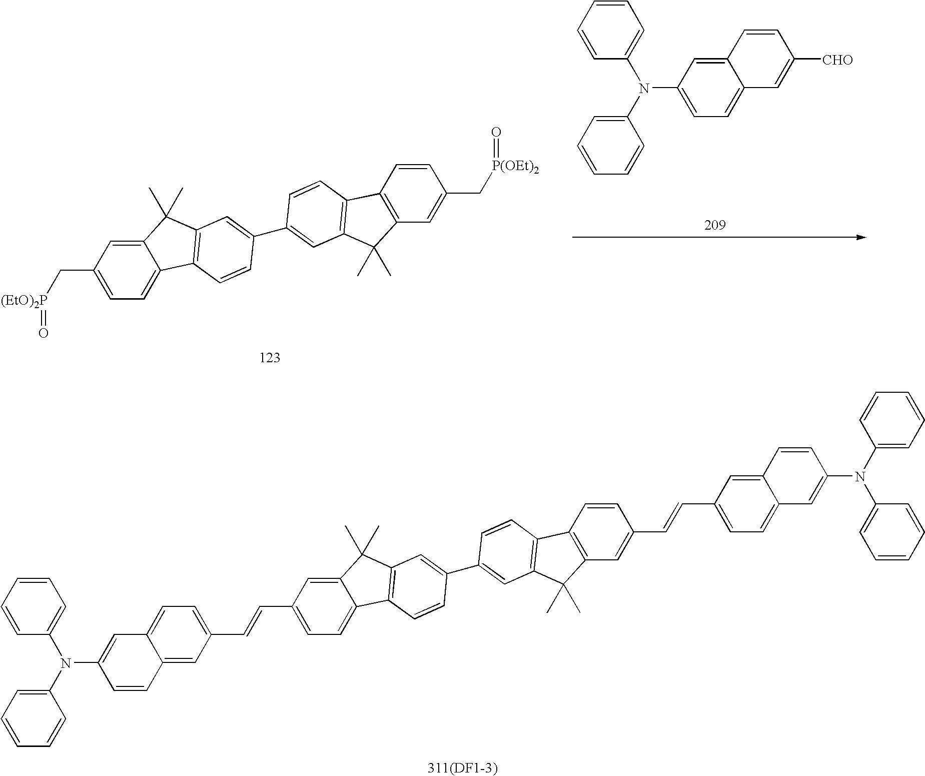

Compound (311, DF1-3) (0.56 g, 0.55 mmol) as the title compound was obtained in total yield of 15.9% according to the same procedure as Synthetic Example 9, but Compound 123 (1 g, 1.46 mmol), and Compound 209 (1.03 g, 3.19 mmol) instead of Compound 205 were employed.

¹H NMR(200 MHz, CDCl₃): δ 1.65(s, 12H), 6.45-6.5(m, 8H), 6.6-6.65(m, 4H), 6.75-6.8(m, 4H), 6.95-7.05(m, 12H), 7.35-7.6(m, 10H), 7.7-7.8(t, 6H), 7.8-7.9(d, 4H) MS/FAB: 1024(found), 1025.36(calculated)

### Synthetic Example 12

Synthesis of IF3-1

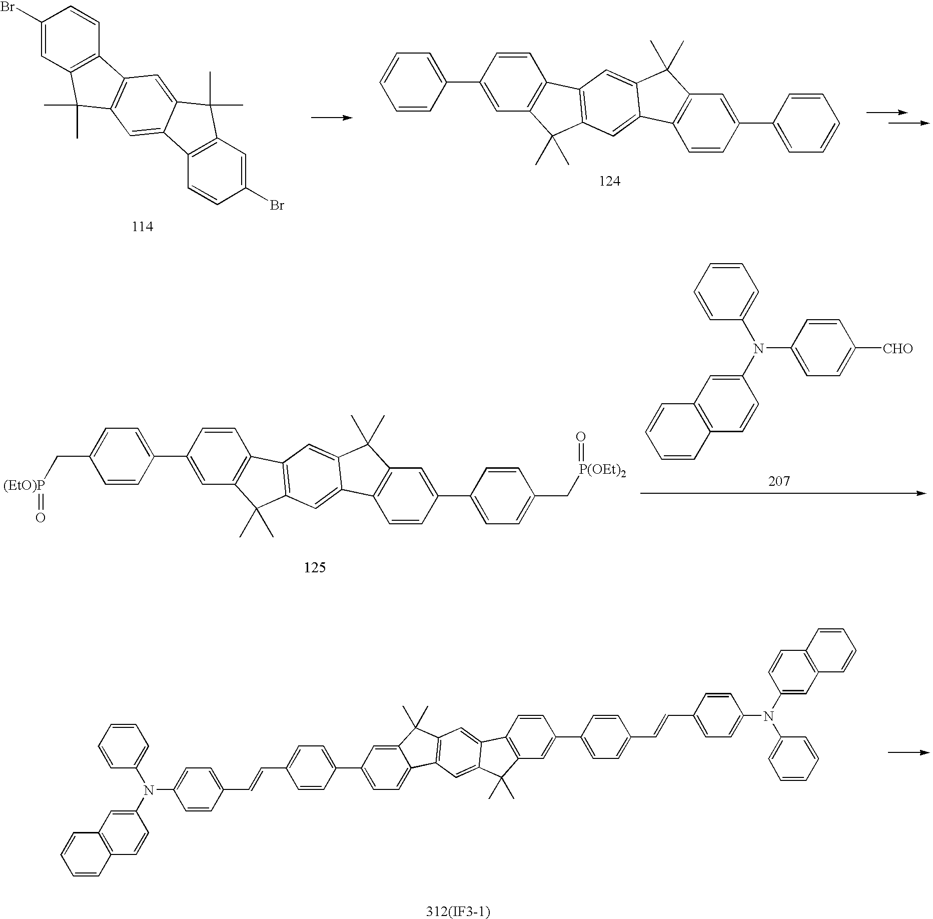

To a solution of Compound (114) (2.0 g, 4.27 mmol) in THF (50 ml), added were phenyl boronic acid (1.10 g, 9.02 mmol), Pd(PPh₃)₄ (1.04 g, 0.90 mmol), and aqueous 2M calcium carbonate solution (15 ml), and the resultant mixture was heated under reflux for 10 hours. From the precipitate thus formed, obtained was Compound (124) (1.80 g, 3.90 mmol).

Compound (124) (1.80 g) and paraformaldehyde (1.7 g) were added to 35% HBr solution in acetic acid (20 ml), and the mixture was heated at 60° C. for 24 hours. Then the temperature of the reaction mixture was lowered to ambient temperature, extracted and dried to obtain a bromomethyl derivative, which was then charged to a reaction vessel. Triethylphosphite (2.57 g, 15.5 mmol) was added thereto at 0° C. and the mixture was heated at 150° C. for 4 hours. After completion of the reaction, residual triethylphosphite was removed by vacuum distillation, and the residue was extracted from ethyl acetate to obtain Compound (125) (1.66 g, 2.18 mmol).

Compound (125) (1.66 g) and Compound (207) (1.48 g, 4.58 mmol) were dissolved in THF (50 ml), and 1.6 M solution of potassium tert-butoxide in THF (8 ml) was added dropwise to the mixed solution at 0° C. The temperature was slowly raised to ambient temperature to complete the reaction. Then an excess amount of water was poured thereto to generate solid, which was then filtered. After recrystallization from ethanol, Compound (312, IF3-1) (1.65 g, 1.65 mmol) was obtained as the title compound in total yield of 38.6%.

¹H NMR(200 MHz, CDCl₃): δ 1.65(s, 12H), 6.45-6.5(m, 8H), 6.6-6.65(m, 2H), 6.75-6.8(m, 4H), 6.95-7.3(m, 16H), 7.4-7.6(m, 14H), 7.65(d, 2H), 7.75(s, 2H), 7.8(s, 2H), 7.95-8.05(d, 2H) MS/FAB: 1100(found), 1101.46(calculated)

### Synthetic Example 13

Synthesis of IF3-2

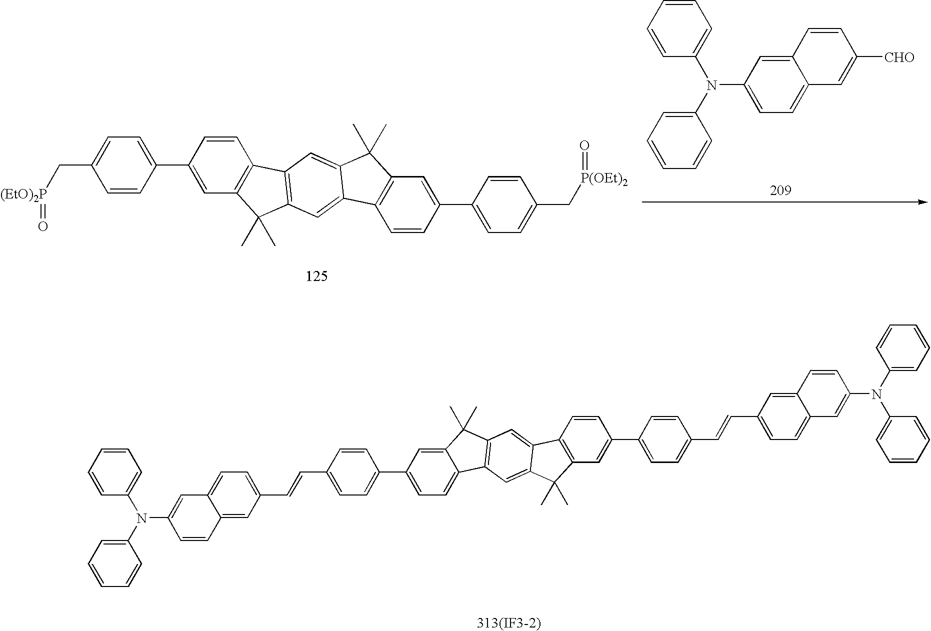

Compound (313, IF3-2) (0.86 g, 0.78 mmol) as the title compound was obtained in total yield of 30.4% according to the same procedure as Synthetic Example 12, but Compound 125 (1 g, 1.31 mmol), and Compound 209 (0.90 g, 2.79 mmol) instead of Compound 207 were employed.

¹H NMR(200 MHz, CDCl₃): δ 1.65(s, 12H), 6.45-6.5(m, 8H), 6.6-6.65(m, 4H), 6.75-6.8(m, 4H), 6.95-7.05(m, 12H), 7.4-7.55(m, 14H), 7.65(d, 2H), 7.7-7.75(d, 4H), 7.8(s, 2H), 7.95-8.05(d, 2H) MS/FAB: 110(found), 1101.46(calculated)

### Synthetic Example 14

Synthesis of DF2-1

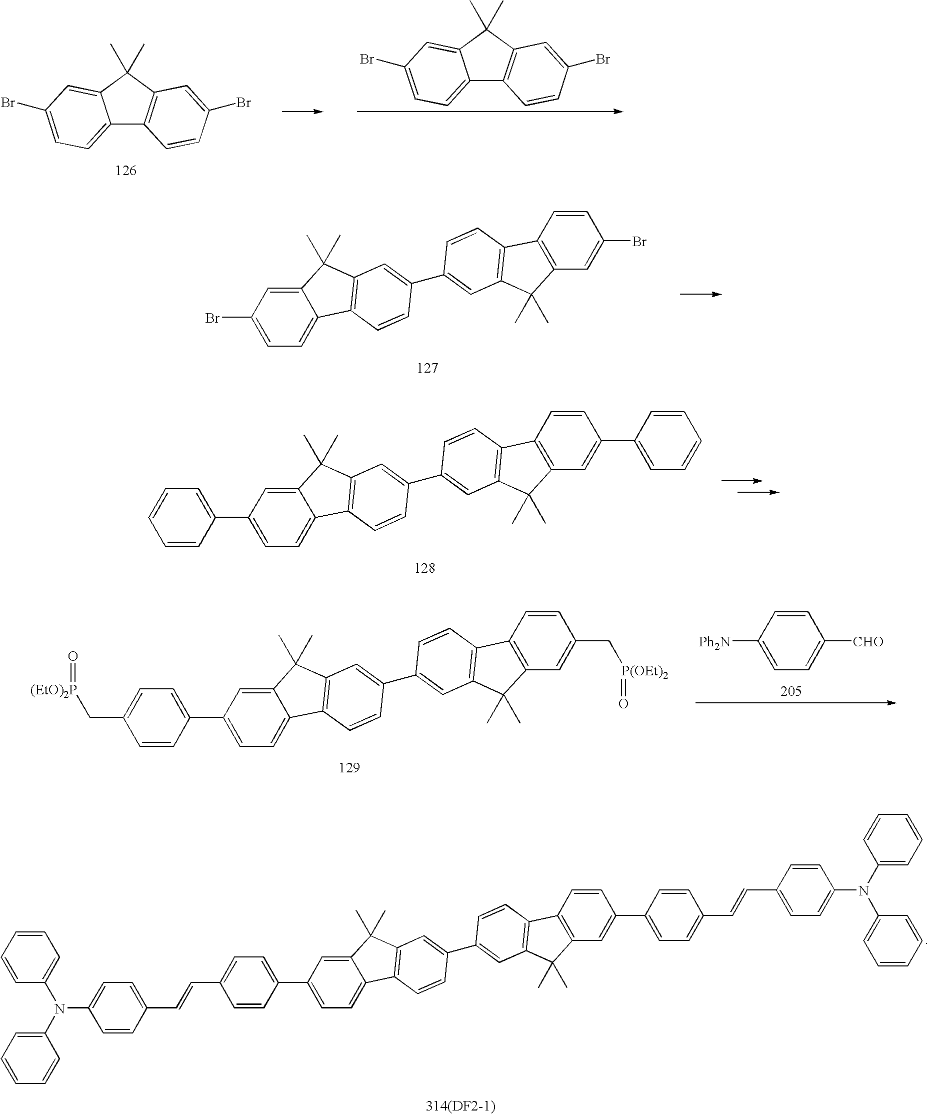

To a solution of 2,7-dibromo-9,9-dimethylfluorene (2.0 g, 5.68 mmol) as Compound (126) in THF (50 ml), added was 1.5 equivalent of tert-butyl lithium at −80° C., and the resultant solution was stirred at −40° C. under nitrogen atmosphere for 12 hours.

After the stirring completed, 2-isopropoxy-4,4,5,5-tetramethyl-1,3,2-dioxaborolane (0.95 g, 5.11 mmol) was added thereto, while maintaining the temperature of the reaction mixture at −80° C. to provide a boronic ester compound of Compound (126) (1.86 g, 4.66 mmol), and the resultant boronic ester compound of Compound (126) (1.86 g) was dissolved in 50 ml of THF. To the solution, added were 2,7-dibromo-9,9-dimethylfluorene (1.56 g, 4.43 mmol), Pd(PPh₃)₄ (0.67 g, 0.58 mmol) and aqueous 2M solution of calcium carbonate (10 ml), and the resultant mixture was heated under reflux for 10 hours. From the precipitate thus formed, obtained was Compound (127) (2.12 g, 3.90 mmol).

To a solution of Compound (127) (2.12 g, 4.27 mmol) in THF (50 ml), added were phenyl boronic acid (1.00 g, 8.20 mmol), Pd(PPh₃)₄ (1.18 g, 1.02 mmol) and aqueous 2M calcium carbonate solution (20 ml), and the resultant mixture was heated under reflux for 10 hours. From the precipitate thus formed, obtained was Compound (128) (1.87 g, 3.48 mmol). Compound (128) (1.87 g) and paraformaldehyde (1.8 g) were added to 35% HBr solution in acetic acid (15 ml), and the mixture was heated at 60° C. for 24 hours. Then the temperature of the reaction mixture was lowered to ambient temperature, extracted and dried to obtain a bromethyl derivative, which was then charged to a reaction vessel. Triethylphosphite (2.30 g, 13.9 mmol) was added dropwise thereto at 0° C. and the mixture was heated at 150° C. for 4 hours. After completion of the reaction, residual triethylphosphite was removed by vacuum distillation, and the residue was extracted from ethyl acetate to obtain Compound (129) (1.32 g, 1.57 mmol).

Compound (129) (1.32 g) and Compound (205) (0.90 g, 3.30 mmol) were dissolved in THF (30 ml), and 1.6 M solution of potassium tert-butoxide in THF (6 ml) was added to the mixed solution dropwise at 0° C. The temperature was slowly raised to ambient temperature to complete the reaction. Then an excess amount of water was poured thereto to generate solid, which was then filtered. After recrystallization from ethanol, Compound (314, DF2-1) (1.28 g, 1.19 mmol) was obtained as the title compound in total yield of 21.0%.

¹H NMR(200 MHz, CDCl₃): δ 1.65(s, 12H), 6.45-6.5(m, 12H), 6.6-6.65(m, 4H), 6.95-7.05(m, 12H), 7.15-7.2(m, 4H), 7.45-7.5(d, 8H), 7.55-7.6(m, 4H), 7.75-7.8(d, 4H), 7.85-7.9(d, 4H)

### Synthetic Example 15

Synthesis of DF2-2

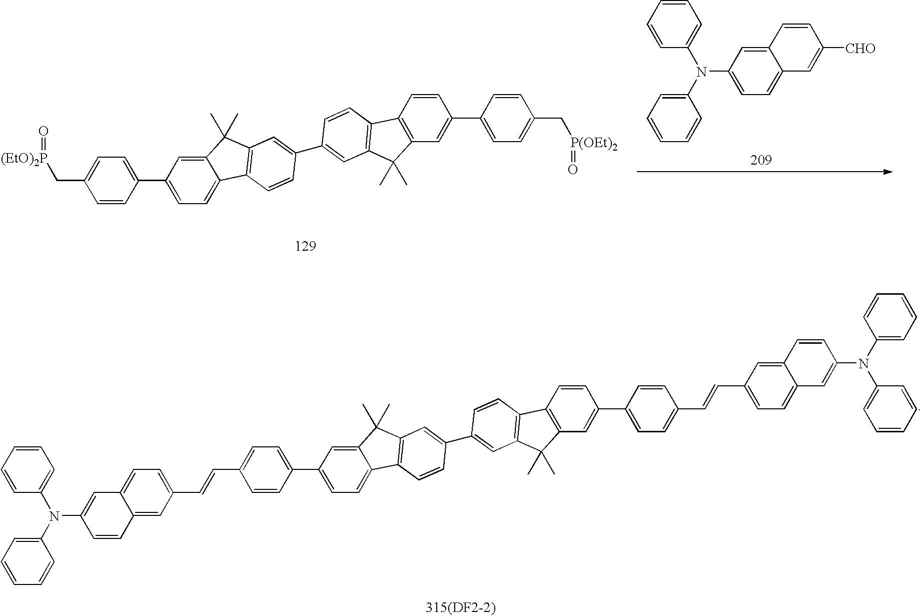

Compound (315, DF2-2) (0.86 g, 0.73 mmol) as the title compound was obtained in total yield of 17.0% according to the same procedure as Synthetic Example 14, but Compound 129 (1.0 g, 1.19 mmol), and Compound 209 (0.93 g, 2.88 mmol) instead of Compound 205 were employed.

¹H NMR(200 MHz, CDCl₃): δ 1.65(s, 12H), 6.45-6.5(m, 8H), 6.6-6.8(m, 8H), 6.95-7.05(m, 12H), 7.35-7.6(m, 18H), 7.7-7.8(t, 6H), 7.85-7.9(d, 4H) MS/FAB: 1176(found), 1177.56(calculated)

### EXAMPLE 1

### Manufacture of OLED Device by Using the Compound According to the Present Invention

An OLED device having the structure employing the electroluminescent material was manufactured.

First, a transparent electrode ITO thin film (15Ω/□) obtained from a glass for OLED was subjected to ultrasonic washing by trichloroethylene, acetone, ethanol and distilled water, subsequently, and stored in isopronanol before use.

Then, an ITO substrate was equipped in a substrate folder of vacuum vapor deposition equipment, and 4,4′,4″-tris(N,N-(2-naphthyl)-phenylamino)triphenylamine (2-TNATA) represented by following structural formula was placed in a cell of the vacuum vapor deposition equipment, which was then ventilated up to 10⁻⁶ torr of vacuum in the chamber. Electric current was applied to the cell to evaporate 2-TNATA to vapor-deposit a hole injection layer having 60 nm of thickness on the ITO substrate.

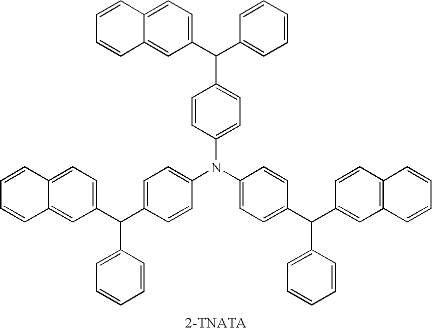

Then, to another cell of the vacuum vapor deposition equipment, charged was N,N′-bis(a-naphthyl)-N,N′-diphenyl-4,4′-diamine (NPB), and electric current was applied to the cell to evaporate NPB to vapor-deposit a hole transport layer of 20 nm of thickness on the hole injection layer.

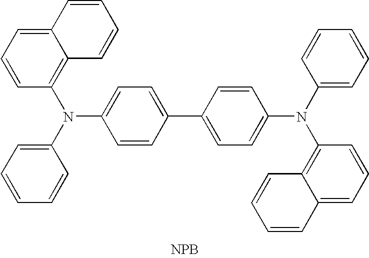

After forming the hole injection layer and hole transport layer, an electroluminescent layer was vapor-deposited thereon as follows. In one cell of the vacuum vapor deposition equipment, charged was dinaphthylanthracene (DNA) represented by following structural formula, and in another cell, an electroluminescent material of a compound according to the present invention (ex. Compound IF2-1), and an electroluminescent layer having 30 nm of thickness was vapor-deposited on said hole transport layer with the vapor deposition rate of 100:1.

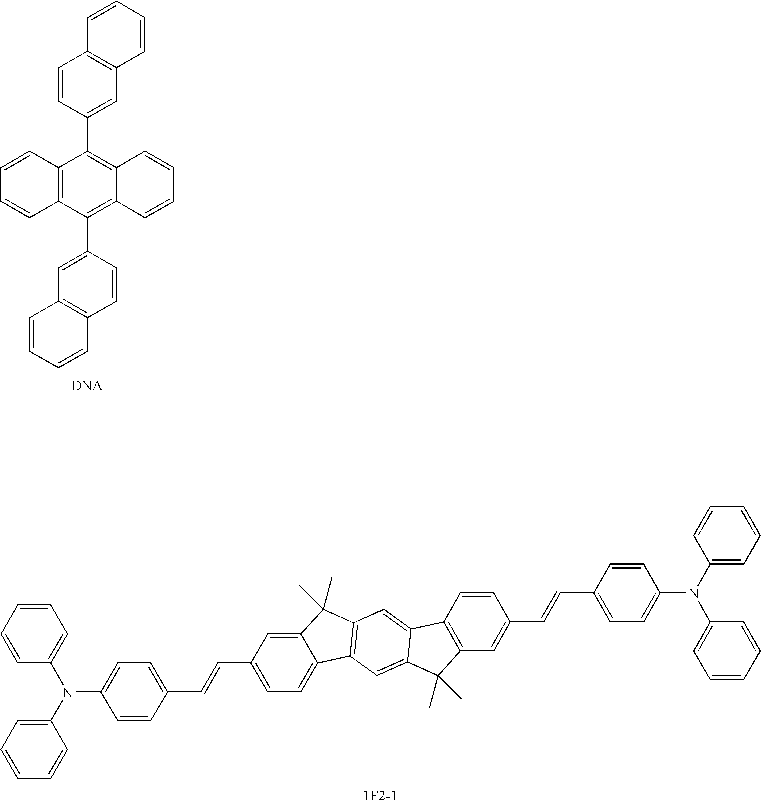

Then, tris(8-hydroxyquinoline)aluminum (III) (Alq) represented by following structural formula was vapor-deposited as an electron transport layer having 20 nm of thickness, and lithium quinolate (Liq) represented by following structural formula was vapor-deposited as an electron injection layer having from 1 to 2 nm of thickness. Thereafter, an Al cathode was vapor-deposited with 150 nm of thickness by using another vapor-deposit device to manufacture an OLED.

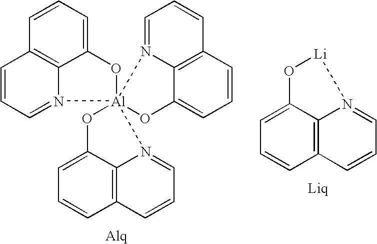

Each material employed in the OLED device was purified by vacuum sublimation under 10-6 torr, and employed as an electroluminescent material for OLED.

### Comparative Example 1

Preparation of an OLED Employing Conventional Electroluminescent Material

A hole injection layer and hole transport layer were created according to the same procedure as described in Example 1, and dinaphthylanthracene (DNA) as a blue electroluminescent material was charged in one cell of said vapor deposition equipment, while perylene having the following structural formula in another cell as another blue electroluminescent material. Then, an electroluminescent layer with 30 nm thickness was vapor-deposited on said hole transport layer with the vapor deposition rate of 100:1.

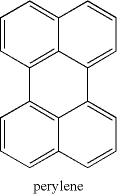

Then, an electron transport layer and an electron injection layer were vapor-deposited according to the same procedure as described in Example 1, and an Al cathode was vapor-deposited by using another vacuum vapor deposition equipment with a thickness of 150 nm, to manufacture an OLED.

### EXAMPLE 2

### Electroluminescent Properties of the OLED Manufactured

Electroluminescent efficiencies of OLEDs comprising the organic electroluminescent compound according to the invention prepared from Example 1 and the conventional electroluminescent compound prepared from Comparative Example 1 were measured at 500 cd/m² and 2,000 cd/m ², respectively, of which the results are shown in Table 1. Since the luminescent properties in the range of low luminance and those applied on a panel are very important in case of blue electroluminescent material, in particular, the data of luminance of about 2,000 cd/m² was established as the standard in order to reflect those properties.

TABLE 1

| | | | | EL Efficiency | | | | |
|---|---|---|---|---|---|---|---|---|
| | EL | EL | | (cd/A) | | Color | | EL |
| | Material | Material | EL peak | @500 | @2,000 | coordinate | | Efficiency/ |
| No. | 1 | 2 | (nm) | cd/m² | cd/m² | X | Y | Y |
| 1 | DNA | IF1-1 | 451 | 3.67 | 2.89 | 0.155 | 0.137 | 26.8 |
| 2 | DNA | IF1-2 | 435 | 2.90 | 2.04 | 0.151 | 0.092 | 31.5 |
| 3 | DNA | IF1-3 | 435 | 3.01 | 2.32 | 0.151 | 0.094 | 32.0 |
| 4 | DNA | IF1-4 | 438 | 2.95 | 2.20 | 0.151 | 0.093 | 31.7 |
| 5 | DNA | IF2-1 | 465 | 11.27 | 12.81 | 0.154 | 0.195 | 65.7 |
| 6 | DNA | IF2-2 | 465 | 11.71 | 12.37 | 0.166 | 0.202 | 61.2 |
| 7 | DNA | IF2-3 | 465 | 10.33 | 12.87 | 0.169 | 0.208 | 61.9 |
| 8 | DNA | IF2-4 | 461 | 8.02 | 10.90 | 0.158 | 0.192 | 56.8 |
| 9 | DNA | DF1-1 | 456 | 7.70 | 8.35 | 0.149 | 0.146 | 57.2 |
| 10 | DNA | DF1-2 | 456 | 7.07 | 8.41 | 0.158 | 0.155 | 54.3 |
| 11 | DNA | DF1-3 | 455 | 6.73 | 7.81 | 0.147 | 0.128 | 61.0 |
| 12 | DNA | IF3-1 | 453 | 6.61 | 6.76 | 0.155 | 0.132 | 51.2 |
| 13 | DNA | IF3-2 | 452 | 5.60 | 5.80 | 0.150 | 0.126 | 44.5 |
| 14 | DNA | DF2-1 | 450 | 5.11 | 5.09 | 0.168 | 0.104 | 49.1 |
| 15 | DNA | DF2-2 | 448 | 4.50 | 4.62 | 0.157 | 0.103 | 44.8 |
| Comp. 1 | DNA | Perylene | 456, 484 | 4.45 | 3.62 | 0.160 | 0.200 | 22.3 |

As can be seen from Table 1, the OLED device employing the organic electroluminescent compounds as the electroluminescent material was compared to the OLED device of Comparative Example which employs widely known DNA:perylene as a conventional electroluminescent material, on the basis of “luminous efficiency/Y” value which shows similar tendency to proton efficiency. As the result, the OLED device employing the organic electroluminescent compound according to the present invention showed higher “luminous efficiency/Y” value than that of Comparative Example.

In view of the fact that the organic electroluminescent compound according to the invention showed higher “luminous efficiency/Y” value, it is found that indenofluorene which is the basic skeletal of the organic electroluminescent compounds of the present invention is a material having high proton efficiency. In addition, it is found that the organic electroluminescent compounds of the present invention can realize higher efficiency and color purity as compared to conventional electroluminescent compounds. In particular, in case of the series of IF2, DF1 and IF3, the “luminous efficiency/Y” value was enhanced 3-folds or more as compared to conventional electroluminescent compound.

In general, in case of a structure comprising a double bond primarily based on stilbene, noticeable enhancement of performances was confirmed. It is assumed that the enhancement of performances in case that the molecular structure wherein the chemical bonds are formed via double bond(s) rather than those wherein only aromatics are simply connected is due to improvement of overlaps between the orbitals of respective aromatic ring in the molecular structure.

From Table 1, it is confirmed that “luminous efficiency/Y” value of the compounds of the invention showed at least two-folds of improvement of the performance as compared to conventional materials, and this comes from the effect of indenofluorene and difluorene skeletal of the present invention to improve by far the valuable properties.

As described above, the organic electroluminescent compounds according to the present invention can be employed as a blue electroluminescent material of high efficiency, thereby having great advantages in terms of luminance of OLED, power consumption and life as compared to conventional ones.

### INDUSTRIAL APPLICABILITY

The organic electroluminescent compounds according to the present invention have good electroluminescent efficiency and excellent life properties, thereby providing OLED having very long lifetime of operation.

1. An organic electroluminescent compound represented by Chemical Formula 1: 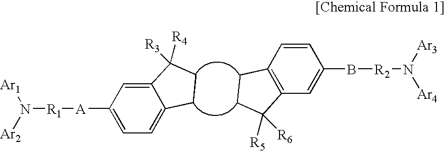
wherein, 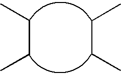
represents 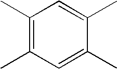
or 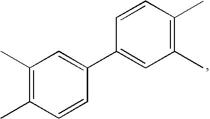
A and B independently represent a chemical bond, 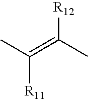
or 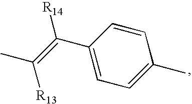
R₁ and R₂ independently represent an aromatic ring or a fused multi-cyclic aromatic ring having two or more aromatic rings,
R₃ through R₆ independently represent a linear or branched C₁-C₂₀ alkyl group with or without halogen substituent(s), a C₅-C₈ cycloalkyl group, or an aromatic group with or without halogen substituent(s),
R₁₁ through R₁₄ independently represent a hydrogen, a C₁-C₈ alkyl group, a C₅-C₈ cycloalkyl group or an aromatic group with or without halogen substituent(s),
Ar₁ through Ar₄ independently represent an aromatic ring or a fused multi-cyclic aromatic ring having two or more aromatic rings.

2. An organic electroluminescent compound according to claim 1, which is represented by one of Chemical Formulas 2 to 6: 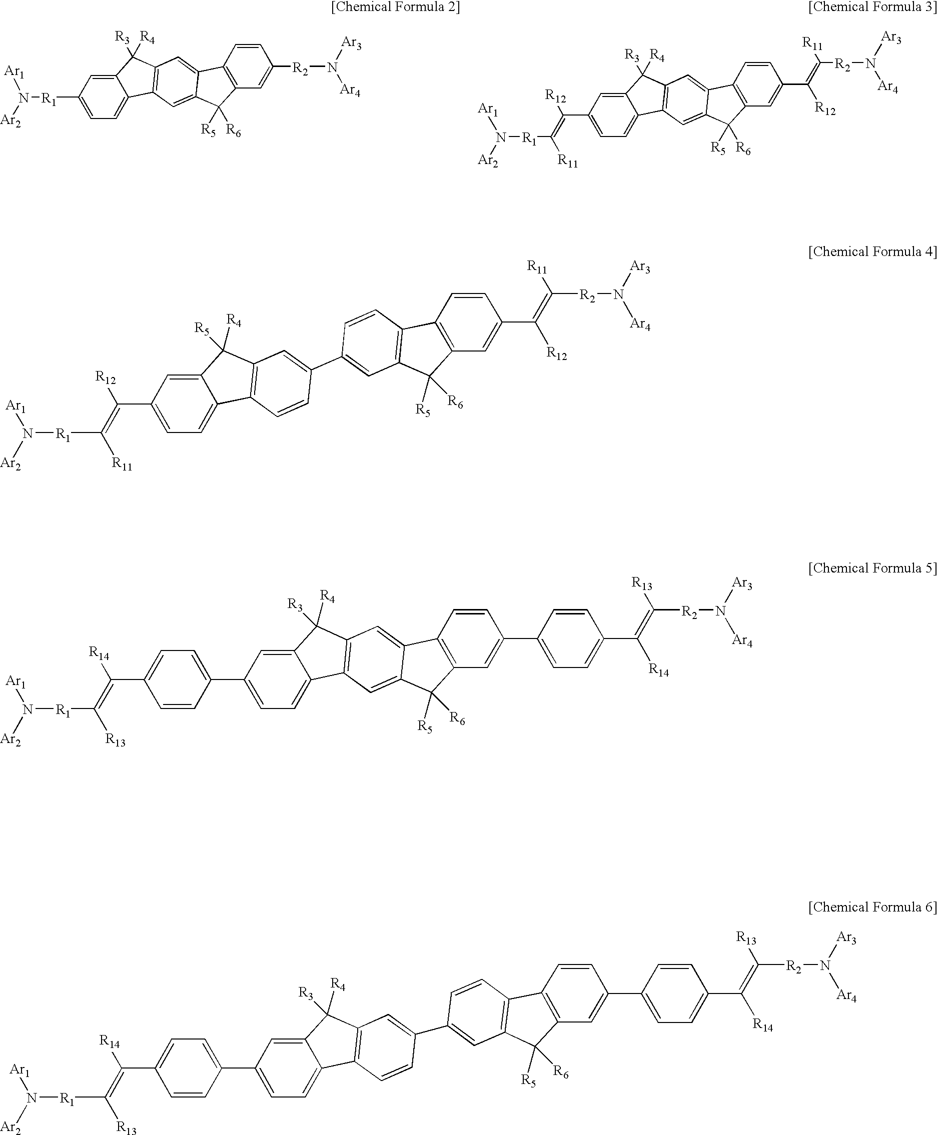
wherein, R₁ and R₂ independently represent an aromatic group or a fused multi-cyclic aromatic ring having two or more aromatic rings,
R₃ through R₆ independently represent a linear or branched C₁-C₂₀ alkyl group with or without halogen substituent(s), a C₅-C₈ cycloalkyl group, or an aromatic group with or without halogen substituent(s),
R₁₁ through R₁₄ independently represent hydrogen, a C₁-C₈ alkyl group, a C₅-C₈ cycloalkyl group or an aromatic group with or without halogen substituent(s), and
Ar₁ through Ar₄ independently represent an aromatic ring or a fused multi-cyclic aromatic ring having two or more aromatic rings.

3. An organic electroluminescent compound according to claim 2, wherein R₁ and R₂ in Chemical Formulas 2 through 6 are selected from phenylene, naphthalene, anthracene, naphthacene, pyrene, fluorene and biphenyl,
R₃ through R₆ represent methyl, ethyl, n-propyl, i-propyl, n-butyl, i-butyl, t-butyl, n-pentyl, i-amyl, n-hexyl, n-heptyl, n-octyl, 2-ethylhexyl, n-nonyl, trifluoromethyl, pentafluoroethyl, cyclopentyl, cyclohexyl, cycloheptyl, cyclooctyl, phenyl, tolyl, 1-naphthyl, 2-naphthyl, 2-fluorophenyl or 4-fluorophenyl,
R₁₁ through R₁₄ represent a hydrogen, methyl, ethyl, n-propyl, i-propyl, n-butyl, i-butyl, t-butyl, n-pentyl, n-hexyl, n-heptyl, n-octyl, cyclopentyl, cyclohexyl, cycloheptyl, cyclooctyl, phenyl, tolyl, 1-naphthyl, 2-naphthyl, 2-fluorophenyl or 4-fluorophenyl, and
Ar₁ through Ar₄ represent phenyl, tolyl, xylyl, pyridyl, biphenyl, naphthyl, anthryl, phenanthryl, naphthacenyl, acenaphthenyl, pyrenyl, fluorenyl or perylenyl.

4. An organic electroluminescent compound according to claim 3, which is selected from the compounds represented by following chemical formulas: 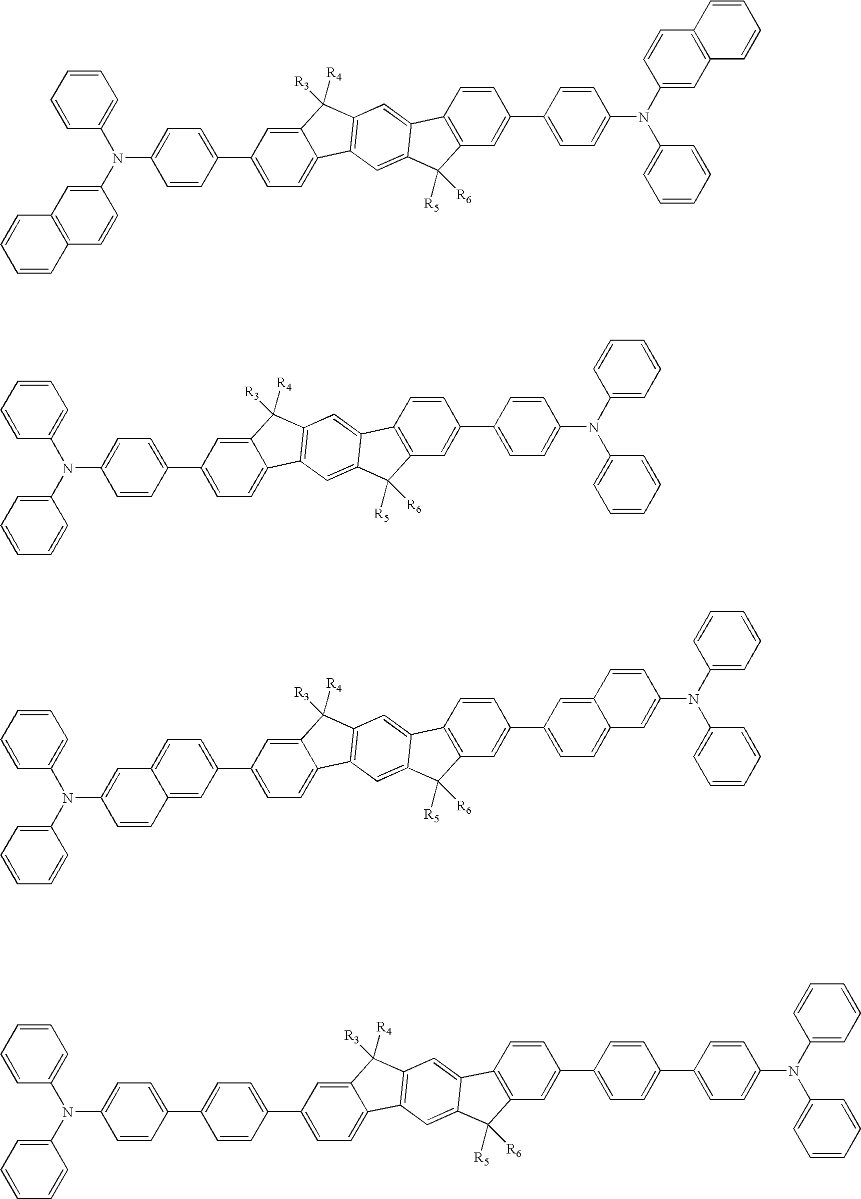 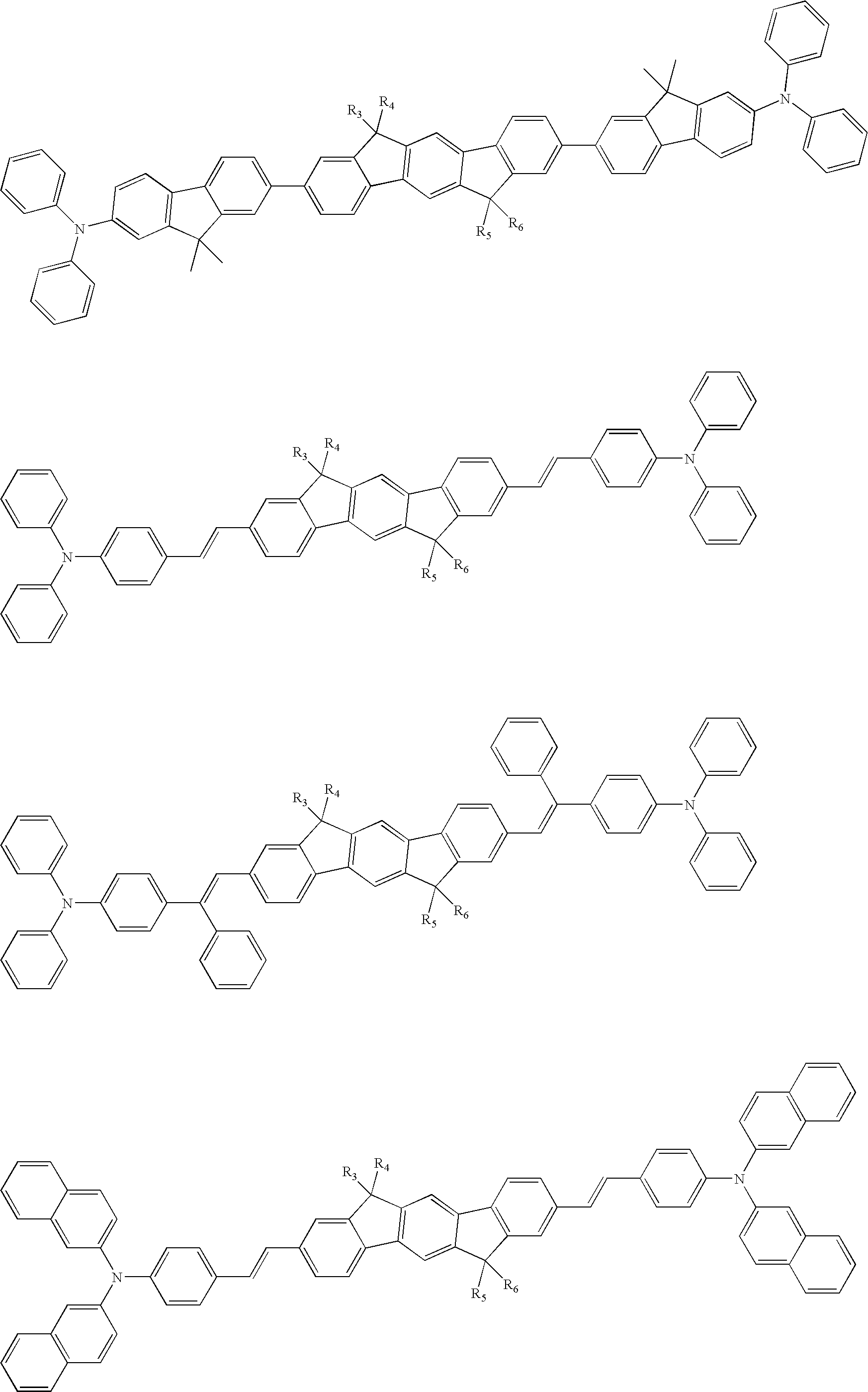 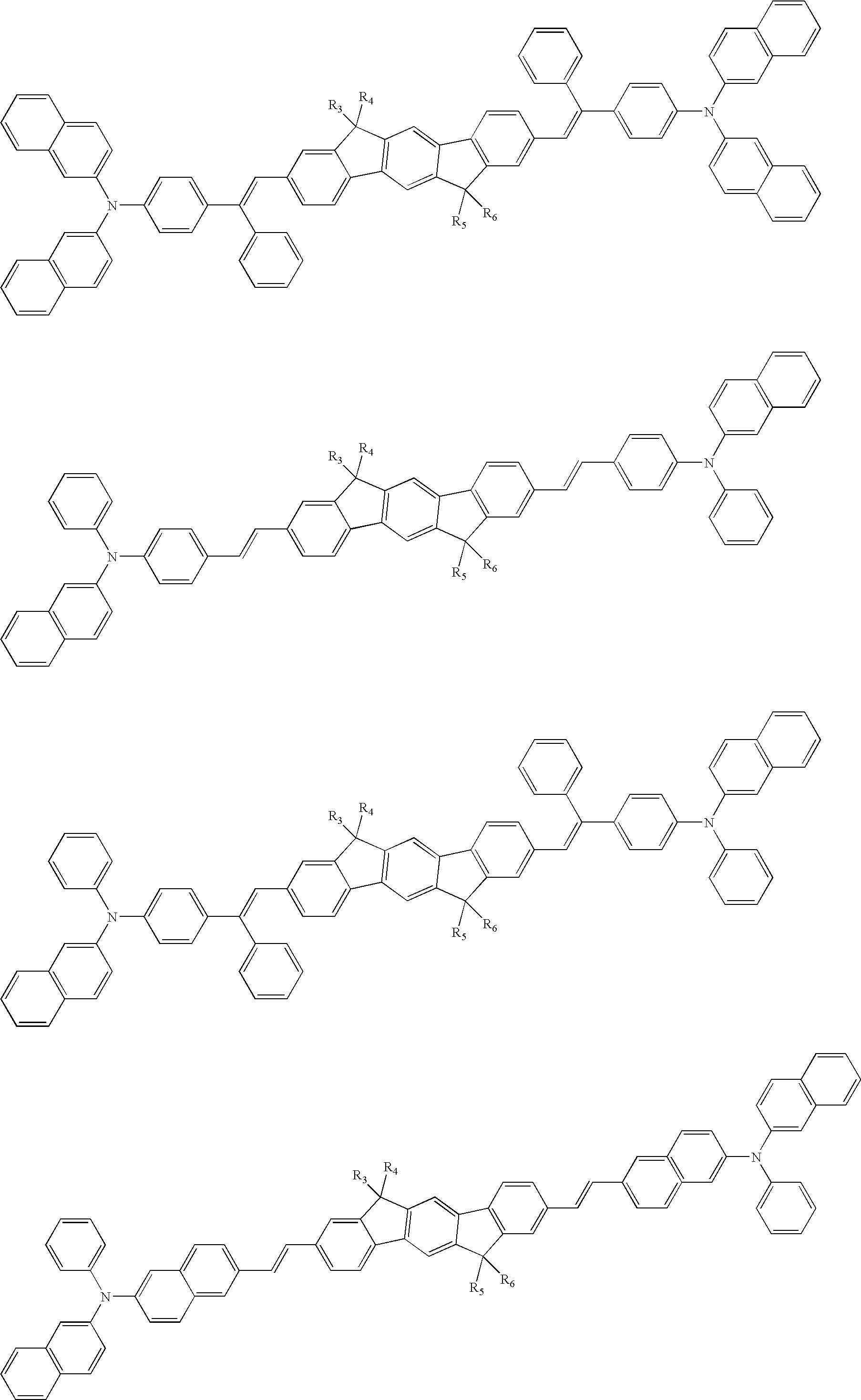 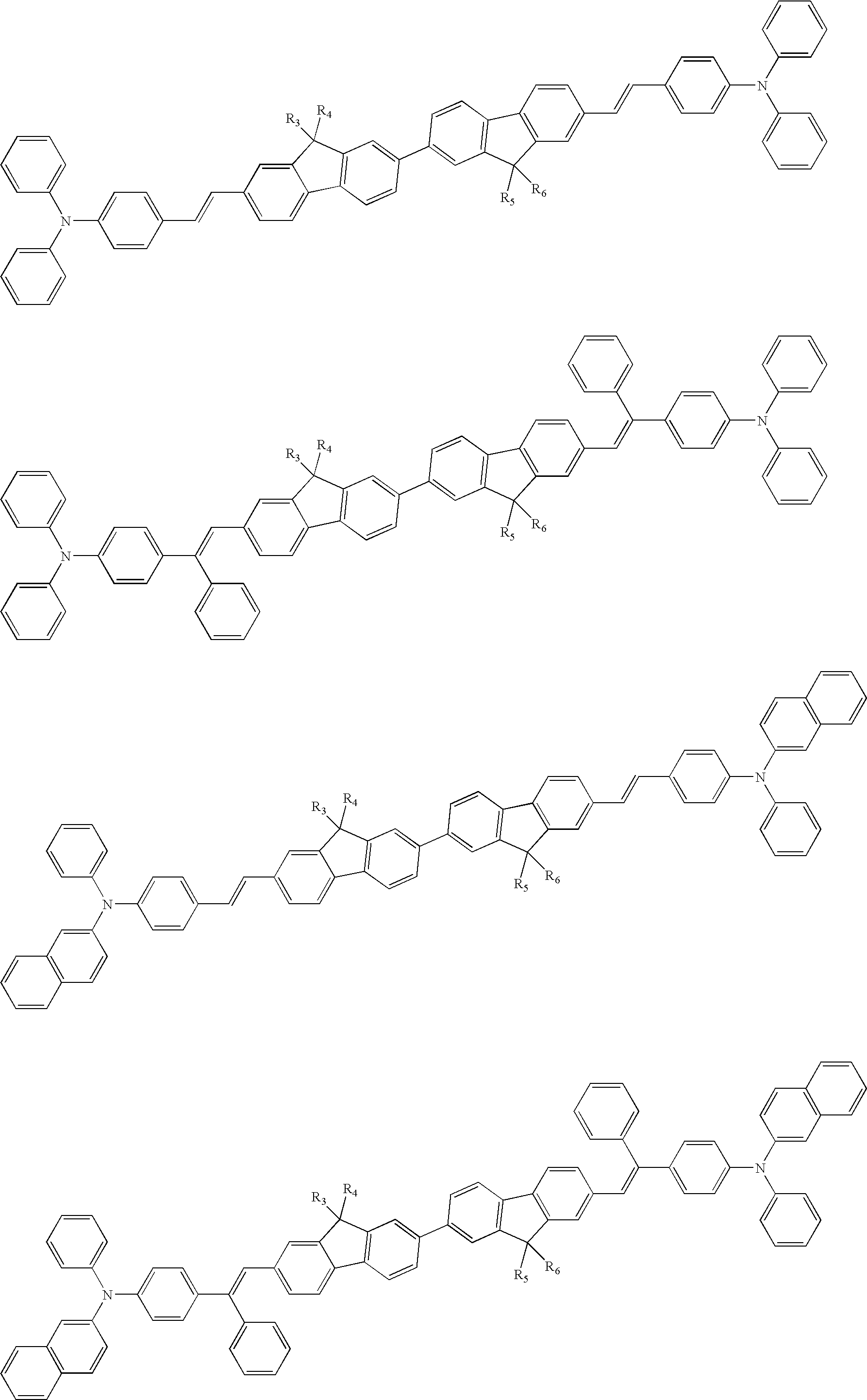 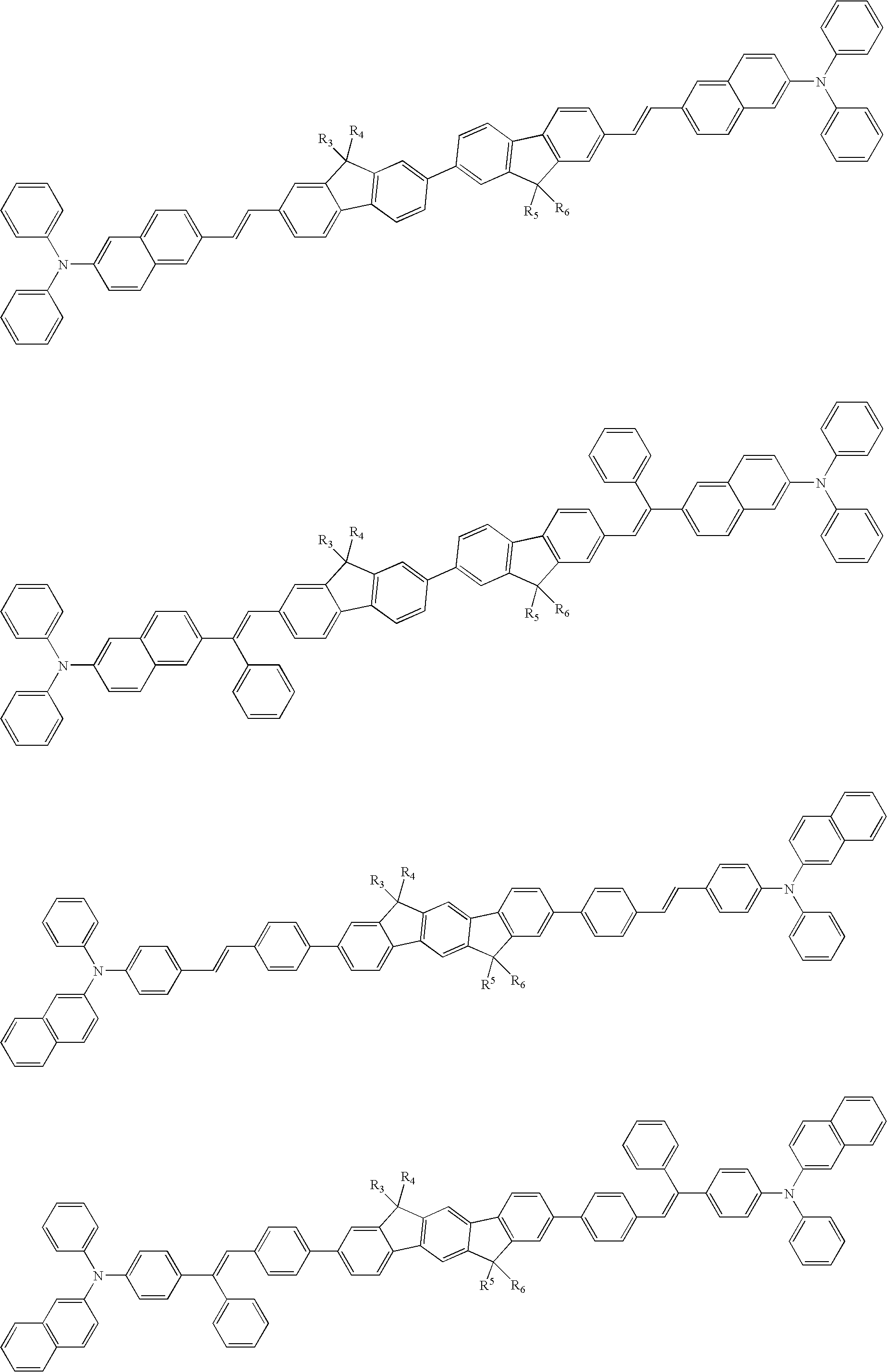 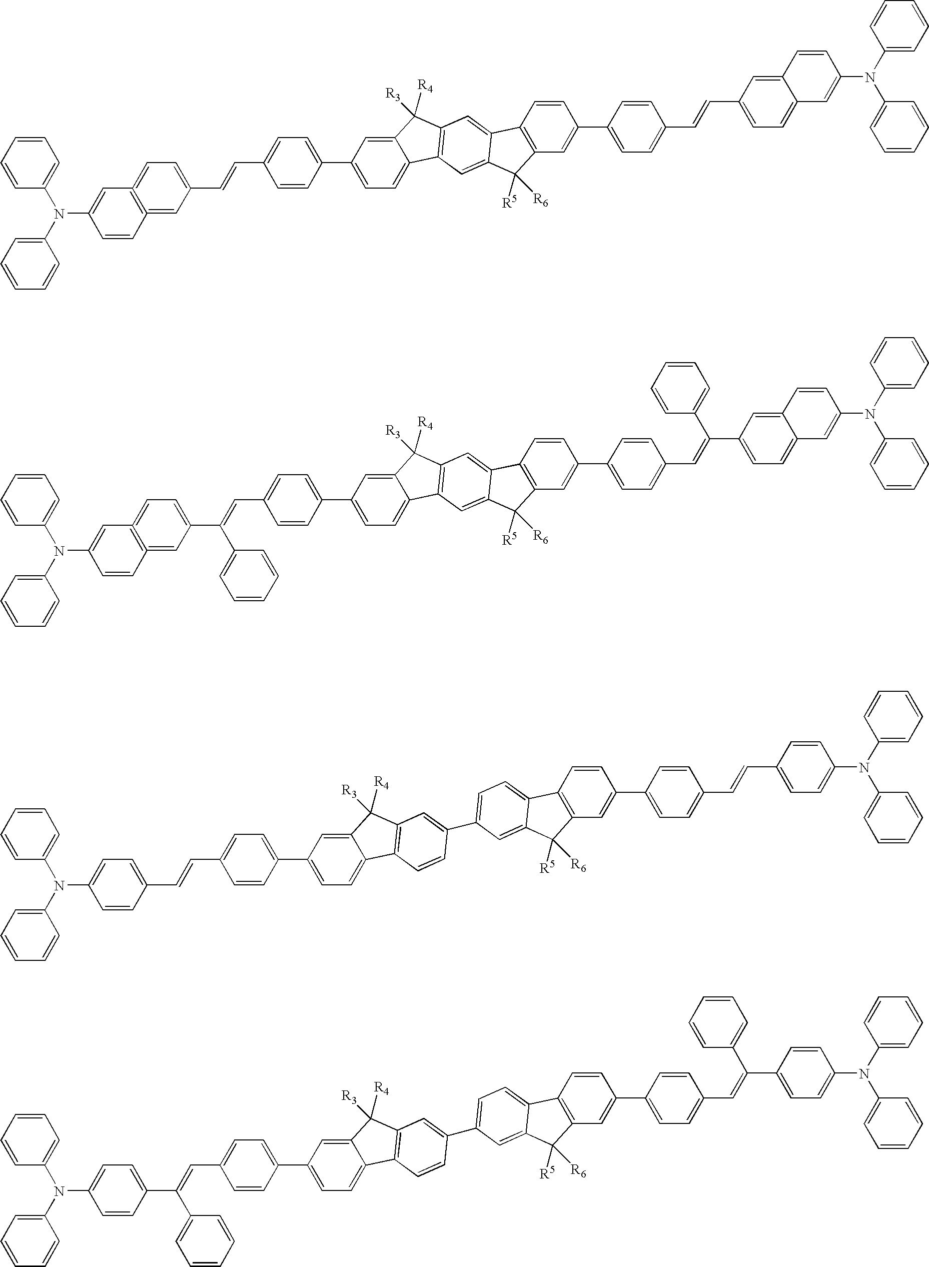 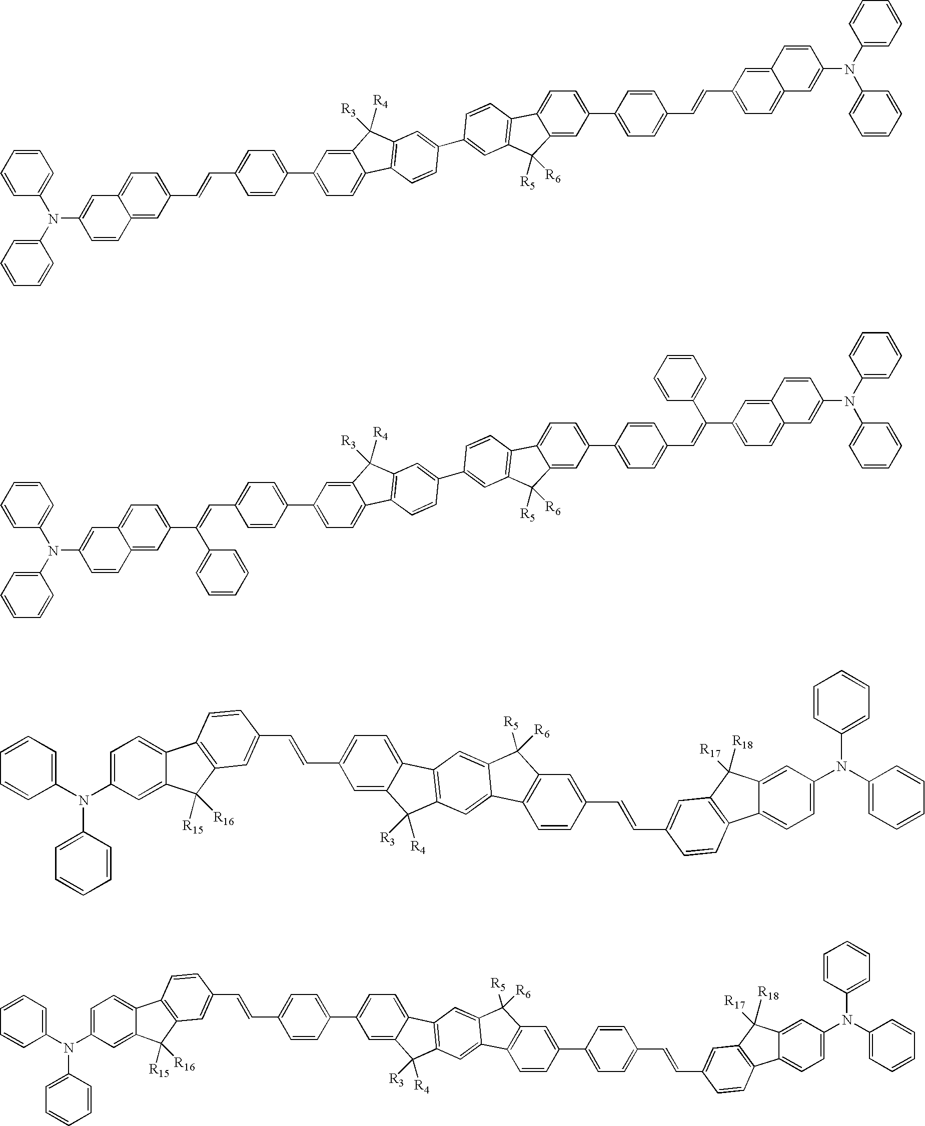 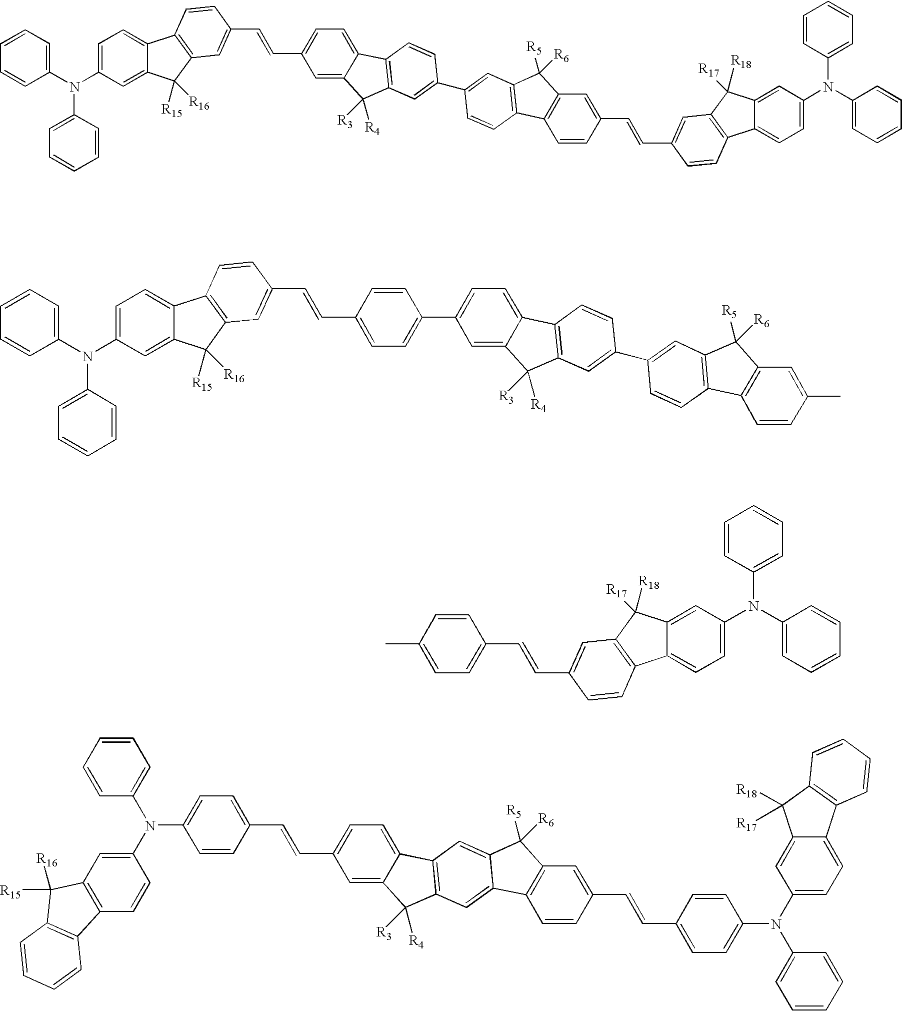 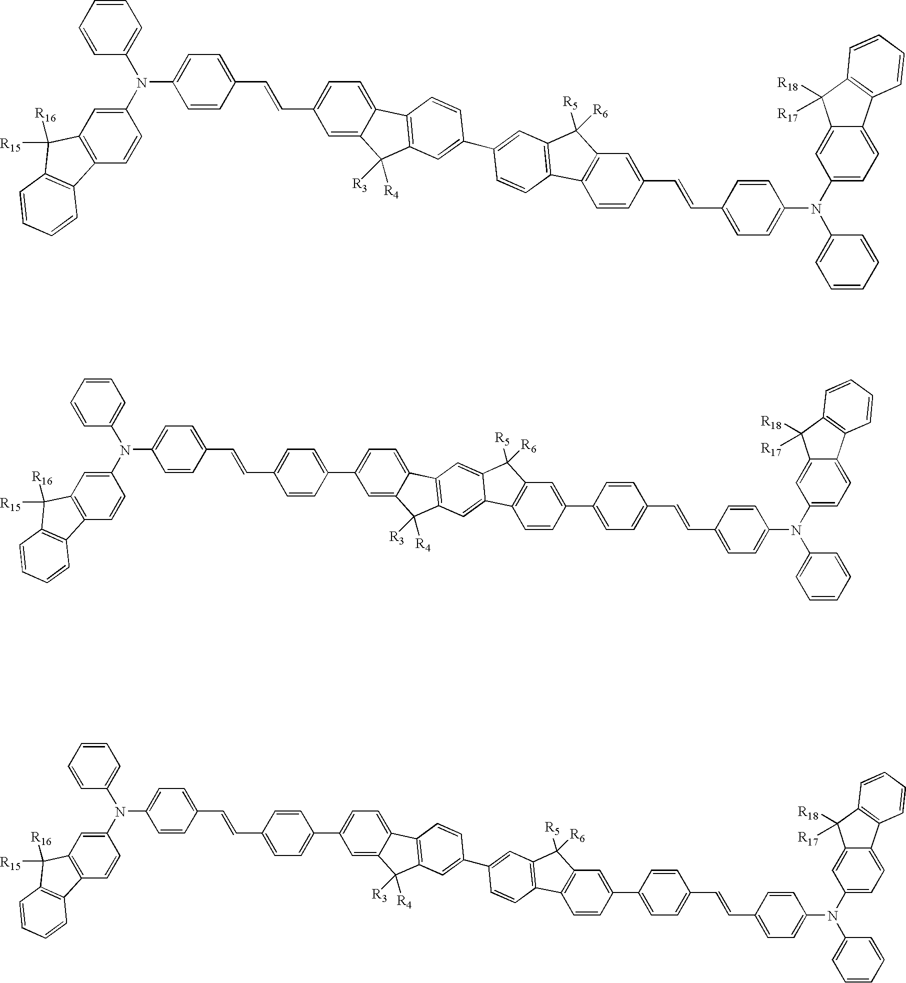
wherein, R₃ through R₆ represent methyl group or ethyl group, and R₁₅ to R₁₈ represent methyl group, ethyl group or phenyl group.

5. An organic electroluminescent device comprising an anode; a cathode; and an organic electroluminescent compound according to claim 1 between the anode and the cathode.

6. An organic electroluminescent device comprising an anode; a cathode; and an organic electroluminescent compound according to claim 2 between the anode and the cathode.

7. An organic electroluminescent device comprising an anode; a cathode; and an organic electroluminescent compound according to claim 3 between the anode and the cathode.

8. An organic electroluminescent device comprising an anode; a cathode; and an organic electroluminescent compound according to claim 4 between the anode and the cathode.
